(19) United States
(12) Patent Application Publication
(10) Pub. No.: US 2026/0174721 A1
(43) Pub. Date: Jun. 25, 2026

(54) AGENT FOR RESTORING METAL HOMEOSTASIS

(71) Applicant: Oxford Antibiotic Group GmbH, Tullln an der Donau (AT)

(72) Inventors: Miroslav GENOV, Tulln (AT); Alexander PRETSCH, Großmugl (AT); Dagmar PRETSCH, Großmugl (AT)

(21) Appl. No.: 18/993,796

(22) PCT Filed: Jul. 14, 2023
(86) PCT No.: PCT/EP2023/069736

§ 371 Date: Jan. 13, 2025

(30) Foreign Application Priority Data
Jul. 14, 2022 (EP) 22185041.5

(51) Int. Cl. A61K 31/225; A61K 31/16; A61K 31/22; A61K 31/4453; A61K 31/4468; A61K 31/47; A61K 31/472; A61K 31/495; A61K 31/5375; A61K 31/5377; A61P 25/16; A61P 25/28; C07C 69/013; C07C 235/74; C07D 211/58; C07D 215/08; C07D 217/06; C07D 295/185

(52) U.S. Cl. CPC A61K 31/225; A61K 31/16; A61K 31/22; A61K 31/4453; A61K 31/4468; A61K 31/47; A61K 31/472; A61K 31/495; A61K 31/5375; A61K 31/5377; A61P 25/16; A61P 25/28; C07C 69/013; C07C 235/74; C07D 211/58; C07D 215/08; C07D 217/06; C07D 295/185

(57) ABSTRACT

The disclosure relates to malonic acid and derivatives thereof of formula (I) below for use as an agent for promoting metallothionein biosynthesis in a patient:

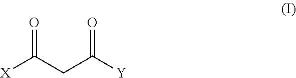

X and Y are each independently selected from —O⁻M⁺, —OR₁, and —NR₂R₃, M⁺ is selected from monovalent or polyvalent metal cations, and R₁, R₂ and R₃ are each independently selected from hydrogen and saturated or unsaturated, linear, branched or cyclic hydrocarbon radicals having from 1 to 25 carbon atoms, and one or more carbon atoms are optionally substituted for O or N, R₂ and R₃ being optionally connected to form a heterocyclic hydrocarbon radical together with the nitrogen atom. The disclosure also relates to a pharmaceutical composition containing a compound of formula (I).

The present invention relates to new drugs for restoring metal homeostasis that are potential agents against neurodegenerative diseases.

### STATE OF THE ART

Neurological diseases such as Alzheimer's, Parkinson's, Huntington's and prion diseases are age-related neuronal diseases characterized by the accumulation of misfolded proteins and neuronal cell death. All of these diseases are currently among the greatest challenges in medicine and occur worldwide.

With almost 50 million cases in 2020, Alzheimer's disease is the leading one of these diseases. Every 3 seconds, a person on this planet develops dementia, of which around 70% are confirmed as Alzheimer's. In general, Alzheimer's disease is a type of dementia that affects the patient's thinking, behavior and memory. The progression of the disease is moderate and reduces the neurons associated with the learning part of the cerebrum. All current approaches for treating Alzheimer's disease provide only temporary symptomatic relief and do not inhibit or reverse the underlying disease mechanisms, as they have been developed mainly on the basis of the amyloid cascade hypothesis, which has not led to a satisfactory cure.

The second major neurological disease of our time is Parkinson's disease, a progressive neurological disorder caused by the degeneration of dopamine receptors in the basal ganglia. It is the most common form of parkinsonism, a group of disorders characterized by two of the four main signs: bradykinesia, rigidity, tremor and postural instability. According to the Parkinson Disease Foundation, around 10 million people worldwide suffer from Parkinson's disease. This makes Parkinson's disease the second most common neurodegenerative disorder, affecting around 1.2% of the world's population over the age of 70. As with Alzheimer's, there are still no available treatments that are able to cure Parkinson's disease. Consequently, the current aim of therapies is to alleviate the symptoms. More than 40 years after its introduction, levodopa (L-DOPA, L-3,4-dihydroxyphenylalanine) of the following formula remains the most effective therapy for reducing the symptoms associated with Parkinson's disease.

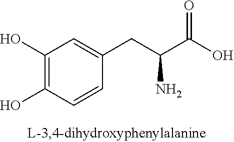 [SMILES: N[C@@H](Cc1ccc(O)c(O)c1)C(=O)O]

WO 2017/142855 A1 discloses derivatives of itaconic acid and malonic acid according to the following formulae I and II, respectively:

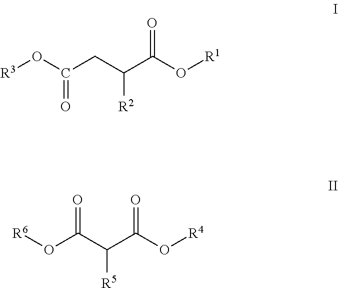

wherein R¹ to R⁶ each independently represent hydrogen or optionally substituted alkyl, alkenyl or alkynyl groups having any number of carbon atoms, the optional substituents of which, in addition to hydroxy, may also comprise an enormous range of sometimes (hetero)aromatic hydrocarbons. These definitions also include derivatives of succinic acid and maleic or fumaric acid, with itaconic acid (also known as methylene succinic acid), malonic acid, their dimethyl esters, as well as fumaric acid and 2-methylfumaric acid (mesaconic acid) being mentioned as preferred.

These compounds are said to act as immunomodulators and, as a result, usable against a multi-faceted list of different types of diseases, including neurodegenerative diseases such as Alzheimer's, Parkinson's, Huntington's and prion diseases. In preferred embodiments, the compounds are said to inhibit the expression of various proteins. In the examples, however, only the free malonic and itaconic acids were investigated for their effect as inhibitors of succinate dehydrogenase, which is said to be based on the structural similarity to their substrate, succinic acid. A potential efficacy in the treatment of neurodegenerative diseases cannot be deduced from this.

In order to overcome this lack of treatment strategies and find other therapeutic options, there has been research on new targets for the treatment of these diseases. In 2008, Bush and Tanzi proposed in their “metal hypothesis of Alzheimer's disease” (A. Bush and R. E. Tanzi, “Therapeutics for Alzheimer's disease based on the metal hypothesis”, Neurotherapeutics 5(3), 421-432 (2008)) that a breakdown in metal homeostasis was the main cause of neurodegenerative diseases, which is why drugs that restore metal homeostasis could provide promising new therapeutic strategies. Subsequently, metal chelators have been repeatedly proposed as potential agents for the treatment of neurodegenerative diseases, including by M. Tosato and A. Di Marco, “Metal Chelation Therapy and Parkinson's Disease: A Critical Review on the Thermodynamics of Complex Formation between Relevant Metal Ions and Promising or Established Drugs”, Biomolecules 9(7), 269 (2019), listing around 800 substances that have been investigated in the years before for the treatment of Parkinson's disease, among which malonic acid is also mentioned as a metal chelator; as well as by Acevedo et al., “Redox active metals in neurodegenerative diseases”, Biol. Inorg. Chem. 24(8), 1141-1157 (2019), who reveal metal chelators as, among other things, a means of preventing misfolding or aggregation of β-amyloid (“Ap”) and α-synuclein, which is one of the main causes of Alzheimer's and Parkinson's disease. Something similar is postulated in a review by P. A. Adlard and A. I. Bush (“Metals and Alzheimer's Disease: How Far Have We Come in the Clinic?”, J. Alzheimer's Dis. 62(3), 1369-1379 (2018)) for the chelating agent clioquinol (5-chloro-7-iodoquinolin-8-ol):

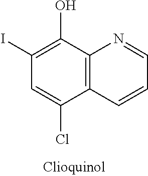 [SMILES: Oc1c(I)cc(Cl)c2cccnc12]

In recent years, the working group of the present inventors has developed a new screening assay using transgenic strains of Caenorhabditis elegans, a nematode from the Rhabditida group, which is based on this new strategy of restoring metal homeostasis, but specifically by upregulating the body's own metallothionein biosynthesis; see Pretsch et al., “Prolongation of metallothionein induction combats Aβ and α-synuclein toxicity in aged transgenic Caenorhabditis elegans”, Sci. Rep. 10, 11707 (Jul. 16, 2020). They describe that by prolonging the endogenous biosynthesis of metallothioneins, i.e., cytoplasmic proteins that bind heavy metals, it was possible to significantly extend the lifespan of corresponding nematode larvae, as this suppresses the expression of toxic β-amyloid (“Aβ”) and α-synuclein. Emodin (1,3,8-trihydroxy-6-methylanthracene-9,10-dione) was identified as one of the agents that achieved this:

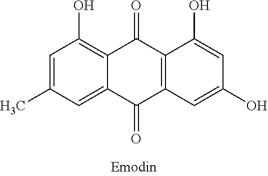 [SMILES: Cc1cc(O)c2c(c1)C(=O)c1cc(O)cc(O)c1C2=O]

A later article (D. Pretsch, “Abnormal metal homeostasis as a common drug target to combat neurodegenerative diseases”, Neural Regen. Res. 16(12), 2388-2389 (2021)) additionally states that not only the effect of emodin, but also that of clioquinol cannot (only) be based on its chelating properties, as other efficient (especially copper) chelates such as D-penicillamine (2-amino-3-mercapto-3-methyl butanoic acid) and diethyldithiocarbamate

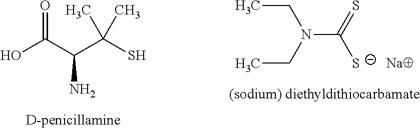

failed in a mouse test model in relation to Parkinson's disease. While D-penicillamine did not show any neuroprotective effect in this study, the presence of diethyl dithiocarbamate even resulted in higher neurotoxicity. The mechanism of action of emodin and clioquinol, which in the C. elegans nematode assay leads to an prolongation of the body's own biosynthesis of metallothioneins, must therefore be independent of the binding of heavy metal ions by chelation.

Hama et al., “Malonic acid suppresses lipopolysaccharide-induced BV2 microglia cell activation by inhibiting the p38 MAPK/NF-κB pathway”, Anim. Cells Syst. (Seoul) 25(2), 110-118 (2021), also disclose malonic acid as a potential treatment for Alzheimer's and Parkinson's disease, however, due to its ability to inhibit the expression of various cytokines, such as interleukins, which can subsequently inhibit various signal transduction pathways involved in the development of inflammation (e.g. the MAPK pathway).

Against this background, the aim of the invention was to use the new assay format based on C. elegans as a model organism to identify chemical compounds that are suitable as agents for promoting the body's own metallothionein biosynthesis and could therefore be new potential active agents against neurodegenerative diseases.

### SUMMARY OF THE INVENTION

The present invention achieves this aim by providing malonic acid and derivatives thereof of formula (I) below for use as an agent for promoting metallothionein biosynthesis in a patient:

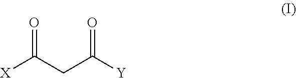

wherein X and Y are each independently selected from —O⁻M⁺, —OR₁, and —NR₂R₃, wherein M⁺ is selected from monovalent or polyvalent metal anions, and R₁, R₂ and R₃ are each independently selected from hydrogen and saturated or unsaturated, linear, branched or cyclic hydrocarbon radicals having from 1 to 25 carbon atoms, wherein one or more carbon atoms are optionally substituted for O or N, R₂ and R₃ being optionally connected to form a heterocyclic hydrocarbon radical together with the nitrogen atom.

Using their recently developed assay format mentioned above, the inventors have surprisingly found that both malonic acid itself and various derivatives of formula (I), namely esters, amides and salts thereof, are effective as agents for promoting metallothionein biosynthesis and thereby restoring metal homeostasis in the C. elegans model organism and could consequently very likely be suitable as agents for combating neurodegenerative diseases. Although unsubstituted malonic acid has a few times been disclosed as an agent for treating neurodegenerative diseases due to its chelating properties, it was not predictable whether or not it—or any of its derivatives—would show a positive effect on the promotion of metallothionein biosynthesis in cells in the nematode assay. This result was particularly surprising for the longer-chain derivatives, some of which are even substituted with aromatic compounds and some of which have relatively bulky structures, which significantly reduces their suitability for the formation of metal chelates due to the limited accessibility of the lone electron pairs of the central nucleophilic moiety C(═O)—CH₂—C(═O).

However, this efficacy was only confirmed for malonic acid derivatives in which the malonic acid moiety is unsubstituted at the central α-carbon atom, which is supported by the later examples and comparative examples, which was also surprising and clearly contradicts that the reason for the efficacy in this assay is merely the ability of the compounds to form chelates with the metal ions. This is also evidenced by the ineffectiveness of compound (108), N-(3-aminopropyl)-N′-tetrahydroquinoline malonic acid diamide, in Comparative Example 8—although all other aromatic-substituted compounds showed positive results.

In one group of embodiments, all of which are salts of malonic acid, at least one of X and Y in formula (I) is —O⁻M⁺, but preferably both X and Y are —O⁻M⁺, wherein M⁺ is independently selected from monovalent and polyvalent metal anions and M⁺ is preferably an alkali metal or alkaline earth metal ion, more preferably an alkali metal ion. Malonic acid salts with only one carboxylate ion as well as those in which both carboxyl groups are present as ionized carboxylate have proven to be correspondingly effective. In the latter case, however, the manufacturing process is somewhat simpler, which is why these are currently to be preferred according to the invention.

Both in the case of only single malonic acid salts, in which only one of X and Y represents —O⁻M⁺ and the other represents an ester group —OR₁ or amide group —NR₂R₃, and in the case of diamides, diesters and ester amides, in which X and Y both represent —OR₁ or NR₂R₃, R₁, R₂ and R₃ are preferably each independently selected from hydrogen and saturated or unsaturated, linear, branched or cyclic hydrocarbon radicals having from 1 to 15 carbon atoms, even more preferably from hydrogen and hydrocarbon radicals having from 1 to 10 carbon atoms, since a lower molecular weight of the compounds reduces the amounts of active substance to be administered.

In particular, the compound according to the present invention is selected from the following: malonic acid (1), malonic acid diamide (2), disodium malonate (3), malonic acid dimethyl ester (4), malonic acid methyl ester potassium salt (5), malonic acid methyl ester amide (6), N-(3-aminopropyl)malonic acid amide (7), malonic acid adamantan-1-yl ester (8), malonic acid adamantan-1-yl ethyl ester (9), malonic acid adamantan-1-yl ester N-(3-aminopropyl)amide (10), malonic acid adamantan-1-yl ester N-(3-aminopropyl)-N-methylamide (11), malonic acid adamantan-1-yl ester piperazine amide (12), malonic acid adamantan-1-yl ester 4-(4-aminobutyryl)piperazine amide (13), malonic acid adamantan-1-yl ester 4-aminopiperidine amide (14), malonic acid adamantan-1-yl ester (4-(4-aminobutyryl)amino)piperidine amide (15), malonic acid methyl ester N-(adamantan-1-yl)amide (16), N-(adamantan-1-yl)malonic acid amide (17), N-adamantan-1-yl-N′-(3-aminopropyl)malonic acid diamide (18), N-adamantan-1-yl-N′-(3-aminopropyl)-N′-methyl malonic acid diamide (19), N-adamantan-1-yl-N′-piperazine malonic acid diamide (20), N-adamantan-1-yl-N′-(4-(4-aminobutyryl)piperazine)malonic acid diamide (21), N-(3-aminopropyl)-N′-morpholine malonic acid diamide (22), N-(3-aminopropyl)-N′-piperidine malonic acid diamide (23), N-(3-aminopropyl)-N′-decahydroquinoline malonic acid diamide (24), N-(3-aminopropyl)-N′-decahydroisoquinoline malonic acid diamide (25), N-(3-aminopropyl)-N′-(6,6-dimethylbicyclo[3.1.1]heptane-2-ylmethyl)malonic acid diamide (26), malonic acid adamantan-1-yl ester N-(3-dimethylaminopropyl)amide (27), malonic acid adamantan-1-yl ester N-(3-morpholinopropyl)amide (28), malonic acid adamantan-1-yl ester 4-(3-trifluoromethylbenzyl)piperazine amide (29), malonic acid adamantan-1-yl ester 4-(3-fluorobenzyl)piperazine amide (30), malonic acid adamantan-1-yl ester 4-(2-methoxyphenyl)piperazine amide (31), malonic acid N-(3,5-dimethyl)adamantan-1-yl amide (32), N-(3,5-dimethyladamantan-1-yl)-N′-(3-aminopropyl)malonic acid diamide (33), N-(3,5-dimethyladamantan-1-yl)-N′-(3-morpholinopropyl)malonic acid diamide (34), N-(3,5-dimethyladamantan-1-yl)-N′-piperazine malonic acid diamide (35), N-(3,5-dimethyladamantan-1-yl)-N′-4-(3-trifluoromethylbenzyl)piperazine malonic acid diamide (36), N-(3,5-dimethyladamantan-1-yl)-N′-4-(3-fluorobenzyl)piperazine malonic acid diamide (37), N-(3,5-dimethyladamantan-1-yl)-N′-4-(2-methoxyphenyl)piperazine malonic acid diamide (38), N-(3,5-dimethyladamantan-1-yl)-N′-4-(4-methoxyphenyl)piperazine malonic acid diamide (39), N-(3,5-dimethyladamantan-1-yl)-N′-4-(2,4-dimethoxyphenyl)piperazine malonic acid diamide (40), and N-(3,5-dimethyladamantan-1-yl)-N′-4-(2-morpholino-2-oxoethyl)piperazine malonic acid diamide (41).

In preferred embodiments, the promotion of metallothionein biosynthesis in a patient serves to restore metal homeostasis and thus to treat a neurodegenerative disease of the patient, which is particularly preferably selected from Alzheimer's disease, Parkinson's disease, and Huntington's disease, and in particular Alzheimer's disease, as according to the “metal hypothesis” cited above, it is likely to be most effectively treatable by restoring metal homeostasis, which should, according to Pretsch et al. (see above), be achievable by promoting the metallothionein biosynthesis.

In a second aspect, the invention accordingly also provides a pharmaceutical composition for therapeutically treating the human or animal body by promoting the metallothionein biosynthesis in a patient, comprising a compound as defined above for use according to the first aspect and at least one pharmaceutically acceptable excipient, and further optionally comprising one or more other pharmaceutically acceptable ingredients, the composition preferably also serving for the treatment of a neurodegenerative disease, more preferably Alzheimer's or Parkinson's disease, in particular Alzheimer's disease.

### EXAMPLES

The present invention is described in more detail below by means of examples and comparative examples which serve to illustrate the invention and are not to be understood as limiting.

The compounds of the examples and comparative examples described below were either purchased commercially or prepared in the manner indicated in the synthesis examples and subsequently tested for their suitability as active agents against neurodegenerative diseases using the assay format disclosed in “Pretsch et al.” mentioned above.

The test compounds were synthesized using standard reaction sequences, wherein all starting products were commercially available or were synthesized in advance from commercially available reagents that were used in the syntheses without further purification. The characterizations were carried out using ¹H NMR spectra recorded using an Avance AV400 spectrometer from Bruker at 400 MHz and mass spectra recorded using an LCMS-8040 from Shimadzu.

Specifically, the compounds below were examined for their effectiveness in this first series of tests. Further tests are currently the subject of the inventors' research work.

As examples of the present invention, the following have been tested so far:

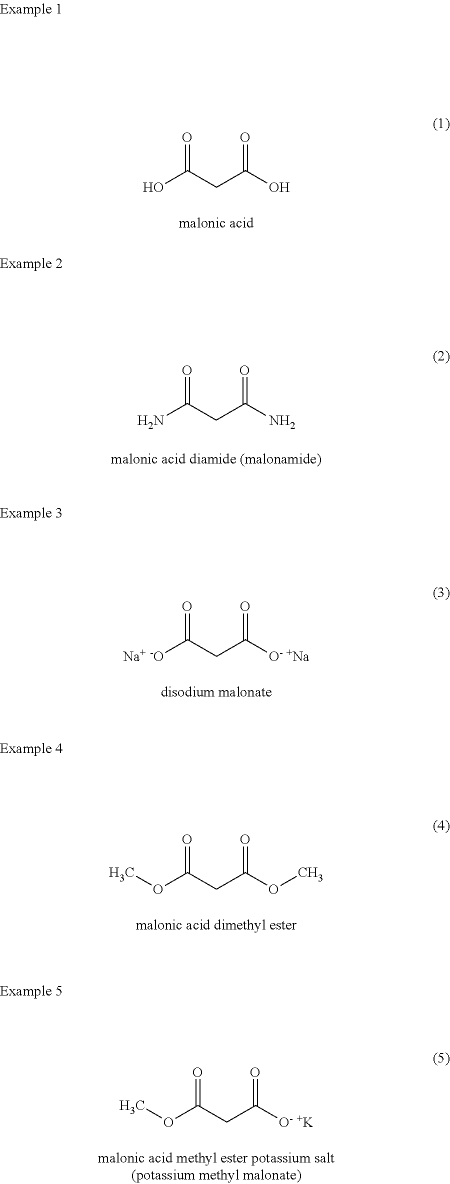

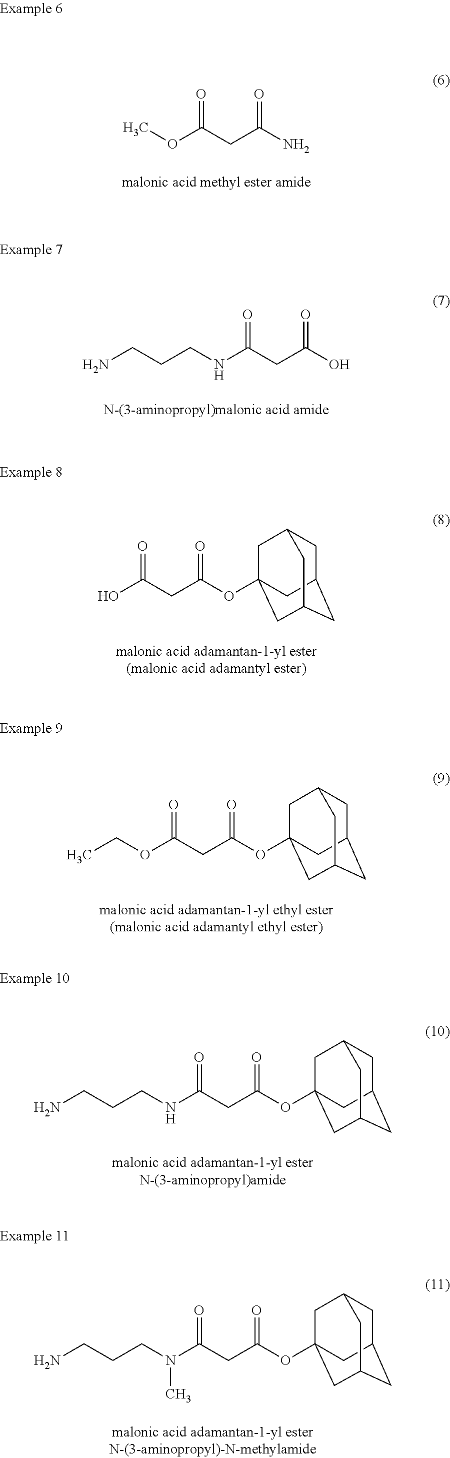

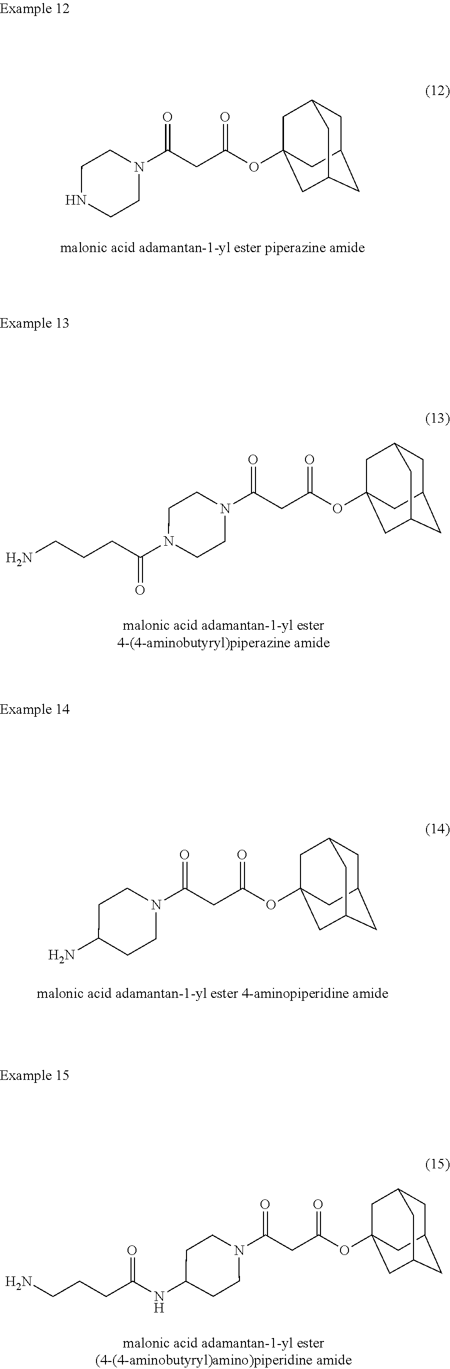 [SMILES: O=C(CC(=O)N1CCNCC1)OC12CC3CC(CC(C3)C1)C2 ; NCCCC(=O)N1CCN(C(=O)CC(=O)OC23CC4CC(CC(C4)C2)C3)CC1 ; NC1CCN(C(=O)CC(=O)OC23CC4CC(CC(C4)C2)C3)CC1 ; NCCCC(=O)NC1CCN(C(=O)CC(=O)OC23CC4CC(CC(C4)C2)C3)CC1]

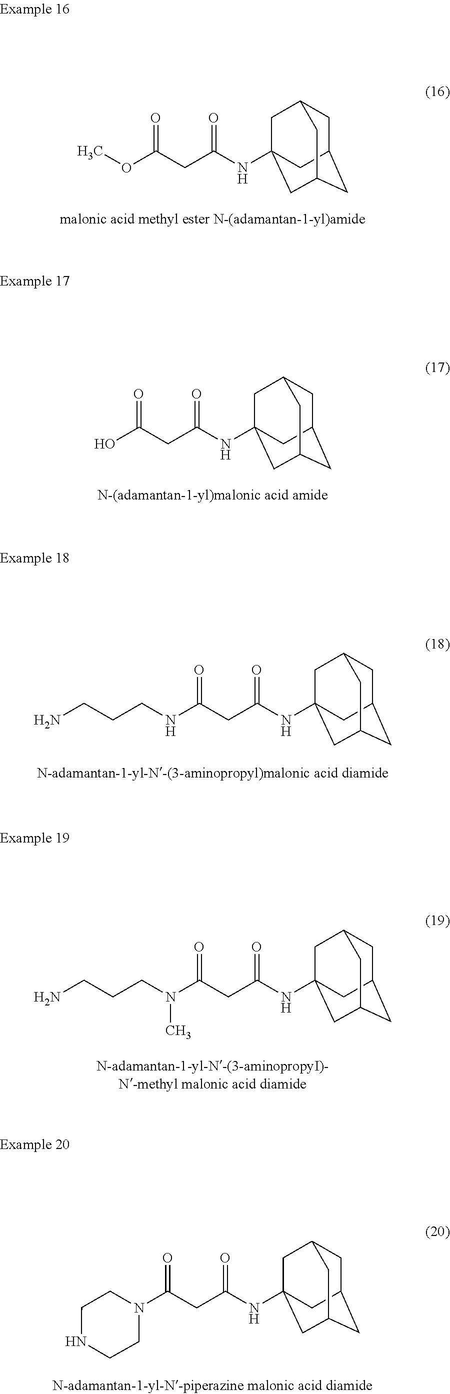 [SMILES: COC(=O)CC(=O)NC12CC3CC(CC(C3)C1)C2 ; O=C(O)CC(=O)NC12CC3CC(CC(C3)C1)C2 ; NCCCNC(=O)CC(=O)NC12CC3CC(CC(C3)C1)C2 ; CN(CCCN)C(=O)CC(=O)NC12CC3CC(CC(C3)C1)C2 ; O=C(CC(=O)N1CCNCC1)NC12CC3CC(CC(C3)C1)C2]

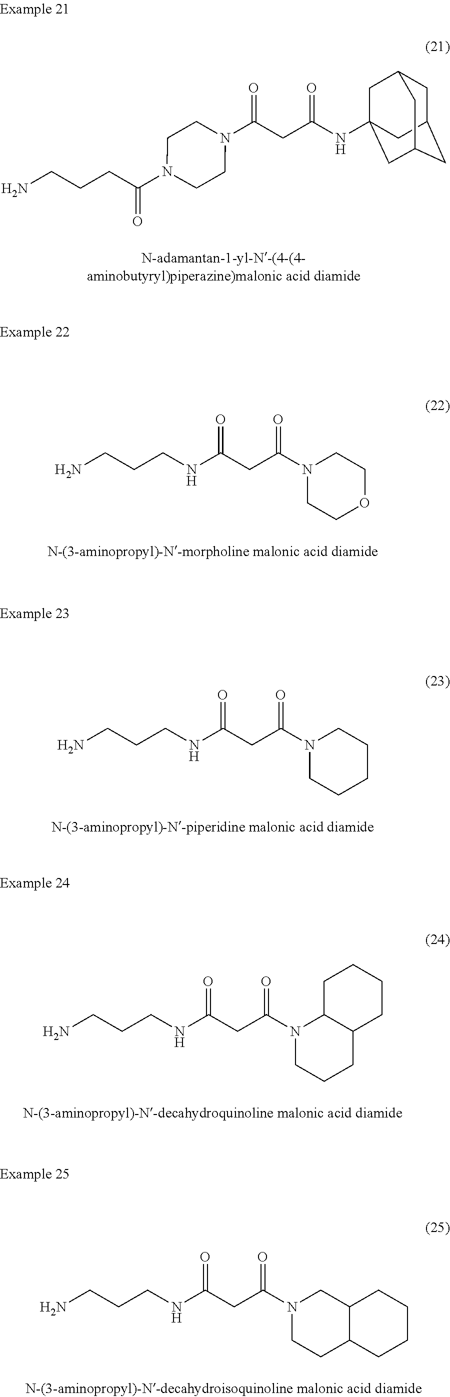 [SMILES: NCCCC(=O)N1CCN(C(=O)CC(=O)NC23CC4CC(CC(C4)C2)C3)CC1 ; NCCCNC(=O)CC(=O)N1CCOCC1 ; NCCCNC(=O)CC(=O)N1CCCCC1 ; NCCCNC(=O)CC(=O)N1CCCC2CCCCC21 ; NCCCNC(=O)CC(=O)N1CCC2CCCCC2C1]

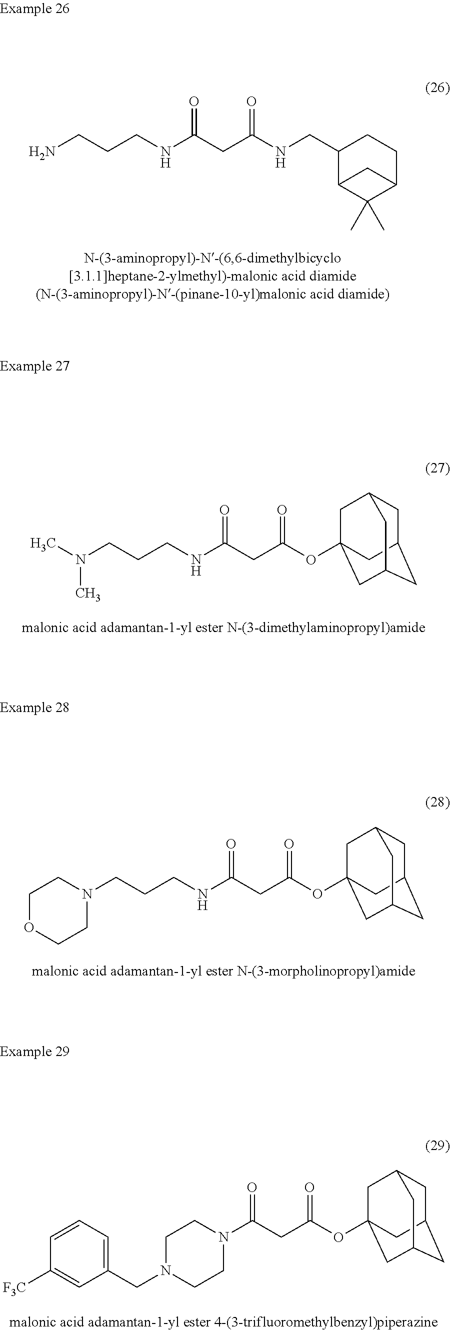 [SMILES: CC1(C)C2CCC(CNC(=O)CC(=O)NCCCN)C1C2 ; CN(C)CCCNC(=O)CC(=O)OC12CC3CC(CC(C3)C1)C2 ; O=C(CC(=O)OC12CC3CC(CC(C3)C1)C2)NCCCN1CCOCC1 ; O=C(CC(=O)N1CCN(Cc2cccc(C(F)(F)F)c2)CC1)OC12CC3CC(CC(C3)C1)C2]

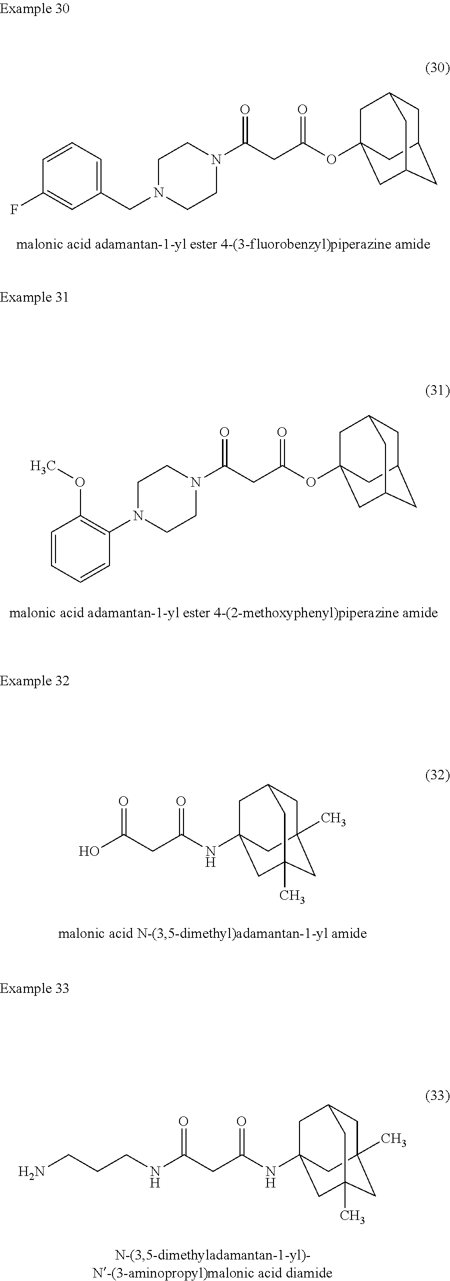 [SMILES: O=C(CC(=O)N1CCN(Cc2cccc(F)c2)CC1)OC12CC3CC(CC(C3)C1)C2 ; COc1ccccc1N1CCN(C(=O)CC(=O)OC23CC4CC(CC(C4)C2)C3)CC1 ; CC12CC3CC(C)(C1)CC(NC(=O)CC(=O)O)(C3)C2 ; CC12CC3CC(C)(C1)CC(NC(=O)CC(=O)NCCCN)(C3)C2]

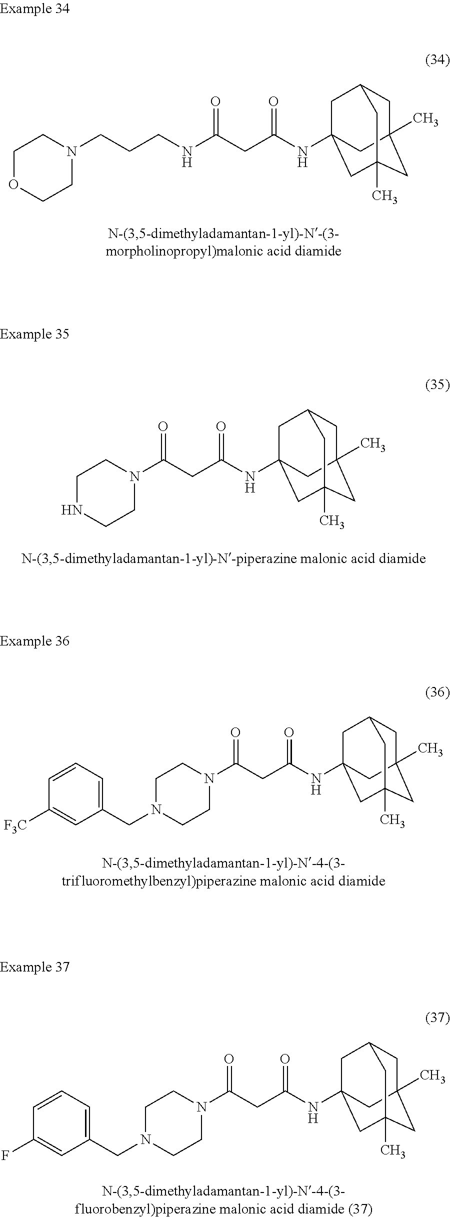 [SMILES: CC12CC3CC(C)(C1)CC(NC(=O)CC(=O)NCCCN1CCOCC1)(C3)C2 ; CC12CC3CC(C)(C1)CC(NC(=O)CC(=O)N1CCNCC1)(C3)C2 ; CC12CC3CC(C)(C1)CC(NC(=O)CC(=O)N1CCN(Cc4cccc(C(F)(F)F)c4)CC1)(C3)C2 ; CC12CC3CC(C)(C1)CC(NC(=O)CC(=O)N1CCN(Cc4cccc(F)c4)CC1)(C3)C2]

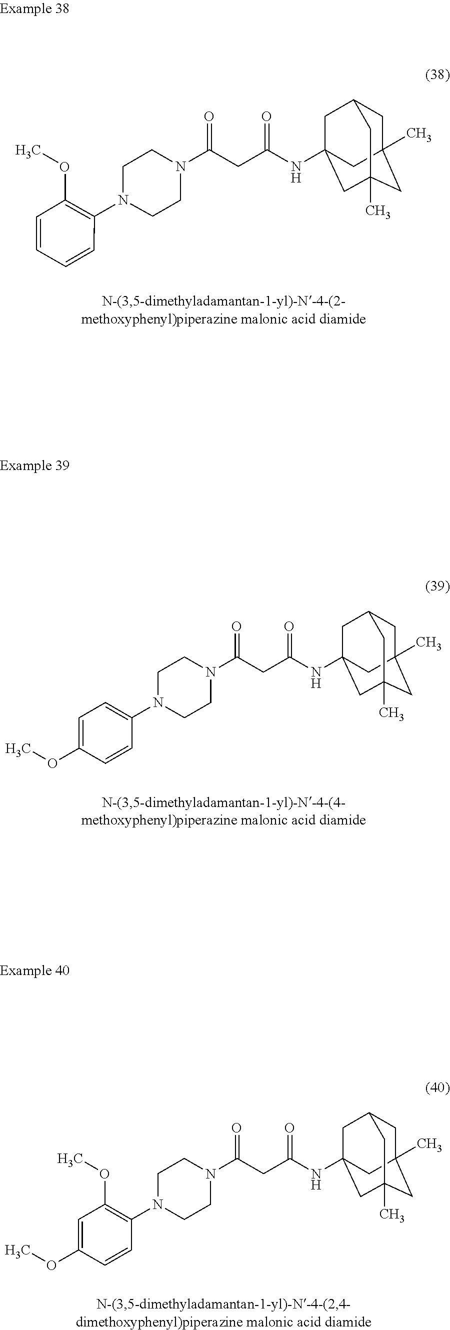

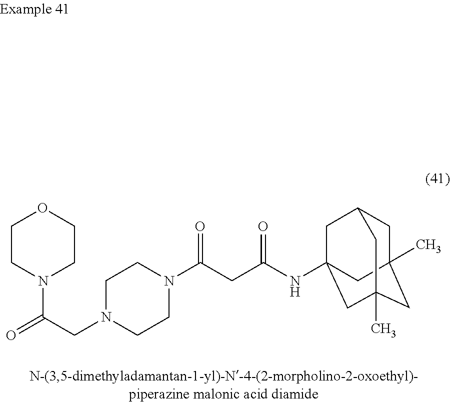 [SMILES: CC12CC3CC(C)(C1)CC(NC(=O)CC(=O)N1CCN(CC(=O)N4CCOCC4)CC1)(C3)C2]

And the following compounds have been examined as comparative examples:

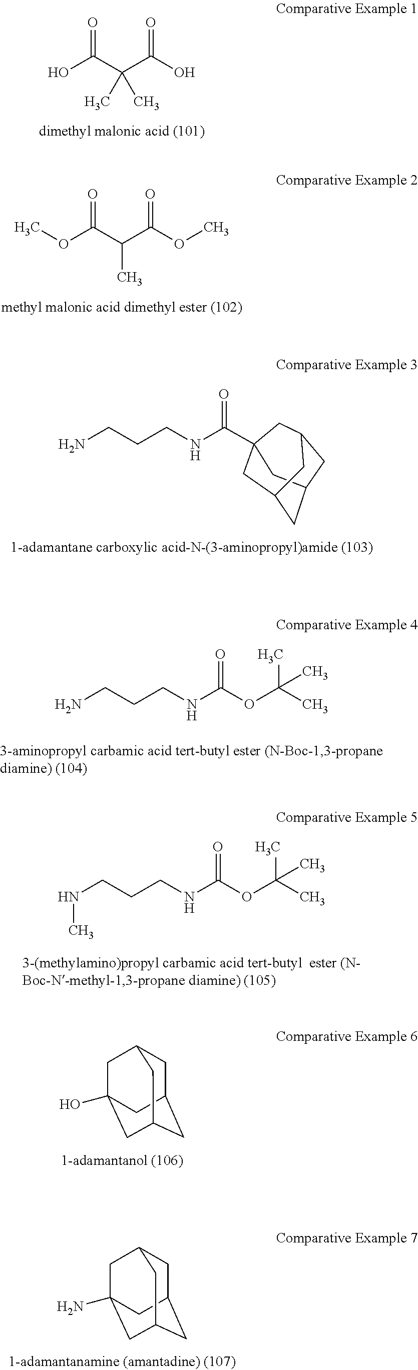

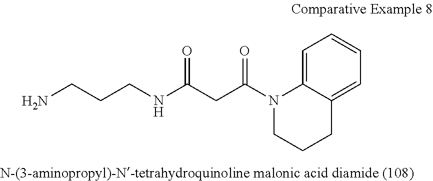 [SMILES: NCCCNC(=O)CC(=O)N1CCCc2ccccc21]

Of these, Compounds (1) to (7) and (101) to (107) were obtained from commercial sources, and the other Compounds (8) to (41) and (108) were prepared as described below.

### Synthesis Example 1

Preparation of malonic acid adamantan-1-yl ester (8)

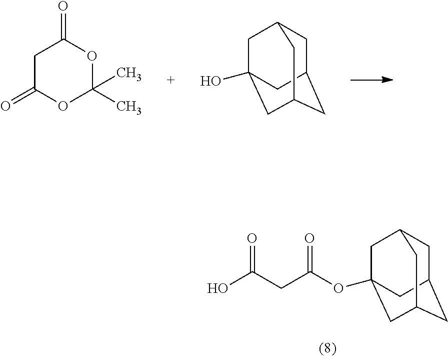

A solution of 1.0 g (6.57 mmol) of adamantan-1-ol and 0.95 g (6.57 mmol) of 2,2-dimethyl-1,3-dioxane-4,6-dione in toluene was refluxed for 12 h, after which the reaction mixture was allowed to cool to room temperature. Subsequently, 60 ml of a saturated aqueous NaHCO₃ solution were slowly added, resulting in phase separation. The toluene phase was discarded, and the aqueous phase was carefully acidified with 100 ml of 1 N HCl. The acidic solution was extracted with dichloromethane, and the organic phase was dried over MgSO₄, filtered and evaporated to dryness, yielding 1.5 g of the title compound as colorless crystals.

¹H NMR (CD₃OD): δ=1.71 (m, 6H, adamantyl), 2.10-2.20 (m, 9H, adamantyl), 3.25 (s, 2H), 4.81 (brs, 1H). MS (ESI−): m/z (%) 237 (100, [M−H]⁻), 283 (47, [M+HCOO⁻]).

### Synthesis Example 2

### Preparation of Malonic Acid adamantan-1-yl Ethyl Ester (9)

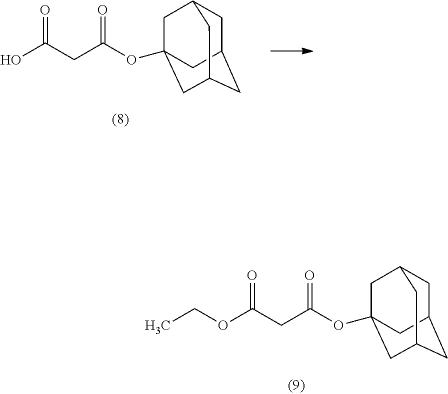 [SMILES: O=C(O)CC(=O)OC12CC3CC(CC(C3)C1)C2 ; CCOC(=O)CC(=O)OC12CC3CC(CC(C3)C1)C2]

To a solution of 35 mg of malonic acid adamantan-1-yl ester (8) in 10 ml ethanol, 2 ml of 4 N HCl in dioxane were added. After stirring for 24 h at room temperature, the reaction mixture was concentrated, and the residue was purified by column chromatography (cyclohexane/ethyl acetate 95:5), yielding the title compound as a colorless liquid (35 mg).

¹H NMR (CDCl₃): δ=1.25 (t, 3H, J=7.14 Hz), 1.62 (m, 6H), 2.08-2.13 (m, 9H), 3.23 (s, 2H), 4.16 (q, 2H, J=7.14 Hz). MS (ESI+): m/z (%) 267 (13, [M+H]⁺), 284 (6, [M+H₂O]⁺), 289 (100, [M+Na]⁺).

### Synthesis Example 3

### Preparation of Malonic Acid adamantan-1-yl Ester N-(3-aminopropyl)amide (10)

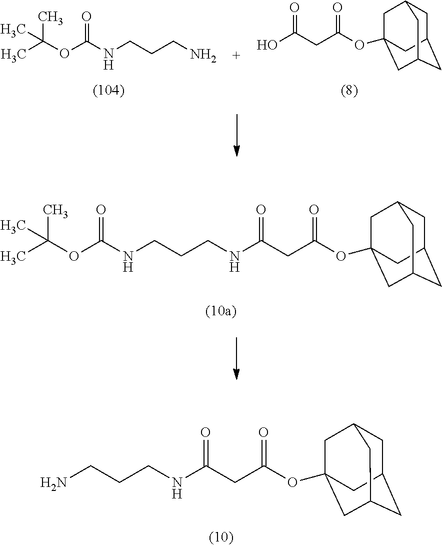 [SMILES: CC(C)(C)OC(=O)NCCCN ; O=C(O)CC(=O)OC12CC3CC(CC(C3)C1)C2 ; CC(C)(C)OC(=O)NCCCNC(=O)CC(=O)OC12CC3CC(CC(C3)C1)C2 ; NCCCNC(=O)CC(=O)OC12CC3CC(CC(C3)C1)C2]

To a solution of 22 mg (0.126 mmol) of 3-aminopropyl carbamic acid tert-butyl ester (N-Boc-1,3-propane diamine) (104) cooled to 0° C., 30 mg (0.126 mmol) of malonic acid adamantan-1-yl ester (8) and 45 μl (0.252 mmol) of diisopropylethylamine (DIPEA) in 8 ml of dimethylformamide (DMF), 54 mg (0.126 mmol) of (1-cyano-2-ethoxy-2-oxoethylideneaminooxy)dimethylamino-morpholino-carbenium hexafluorophosphate (COMU) were added. After stirring for 2 h and allowing to warm to room temperature, the reaction mixture was diluted with ethyl acetate, washed with 1 N HCl and a saturated aqueous NaHCO₃ solution, dried over MgSO₄, filtered and evaporated to dryness. The residue (47 mg) containing the Boc-protected malonic acid adamantan-1-yl ester N-(3-aminopropyl)amide (10a) was used in the next step without further purification.

MS (ESI+): m/z (%) 395 (100, [M+H]⁺), 417 (100, [M+Na]⁺), 811 (100, [2M+Na]⁺).

The residue thus obtained was dissolved in 8 ml of dichloromethane, 1.26 ml of 4 N HCl in dioxane were added, and the mixture was stirred for 2 h at room temperature. Then, the volatiles were removed in vacuo, yielding the hydrochloride of the title compound (10) in the form of a yellow oil (34 mg).

¹H NMR (CDCl₃): δ=1.60-1.72 (m, 10H), 2.10-2.18 (m, 9H), 2.85 (s, 2H), 3.22-3.25 (m, 1H), 3.62-3.70 (m, 2H), 3.75-3.79 (m, 1H). MS (ESI+): m/z (%) 295 (100, [M+H]⁺), 589 (80, [2M+H]⁺), 611 (15, [2M+Na]⁺).

### Synthesis Example 4

### Preparation of Malonic Acid adamantan-1-yl Ester N-(3-aminopropyl)-N-methylamide

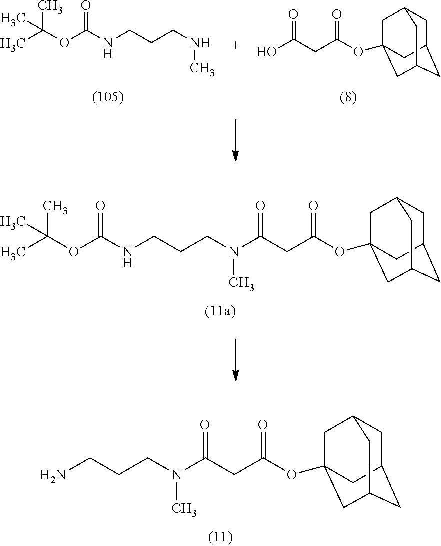 [SMILES: CNCCCNC(=O)OC(C)(C)C ; O=C(O)CC(=O)OC12CC3CC(CC(C3)C1)C2 ; CN(CCCNC(=O)OC(C)(C)C)C(=O)CC(=O)OC12CC3CC(CC(C3)C1)C2 ; CN(CCCN)C(=O)CC(=O)OC12CC3CC(CC(C3)C1)C2]

To a solution of malonic acid adamantyl ester (8) (289 mg, 1.211 mmol), 3-(methylamino)propyl carbamic acid tert-butyl ester (105) (228 mg, 1.211 mmol), and triethylamine (TEA) (340 μl, 2.422 mmol) in DMF (6 ml), 1 ml of a 1.6 M solution of propane phosphonic anhydride (T3P) in ethyl acetate was slowly added and stirred for 1 h at room temperature. The reaction mixture was then mixed with saturated saline for hydrolysis and extracted with dichloromethane. The extract was dried over MgSO₄, filtered and evaporated to dryness. The residue was purified by column chromatography (dichloromethane/ethyl acetate 70:30), yielding the Boc-protected malonic acid adamantan-1-yl ester N-(3-aminopropyl)-N-methylamide (11a) in the form of a colorless oil (370 mg).

MS (ESI+): m/z (%) 409 (100, [M+H]⁺, 431 (100, [M+Na]⁺), 309 (100, [M-Boc]⁺), 839 (100, [2M+Na]⁺).

The product thus obtained (0.96 mmol) was dissolved in 20 ml dichloromethane, 9.6 ml of 4 N HCl in dioxane were added and the mixture was stirred for 1.5 h at room temperature. The volatile components were then removed in vacuo, yielding the hydrochloride of the title compound (11) in the form of a yellow oil (260 mg).

¹H NMR (DMSO-d₆): δ=1.63-1.75 (m, 10H), 2.10-2.20 (m, 9H), 2.85 (s, 2H), 2.97 (s, 3H) 3.22-3.25 (m, 1H), 3.61-3.72 (m, 2H), 3.73-3.82 (m, 1H). MS (ESI+): m/z (%) 309 (100, [M+H]⁺), 331 (17, [M+Na]⁺), 617 (77, [2M]⁺).

### Synthesis Example 5

### Preparation of Malonic Acid adamantan-1-yl Ester Piperazine Amide (12)

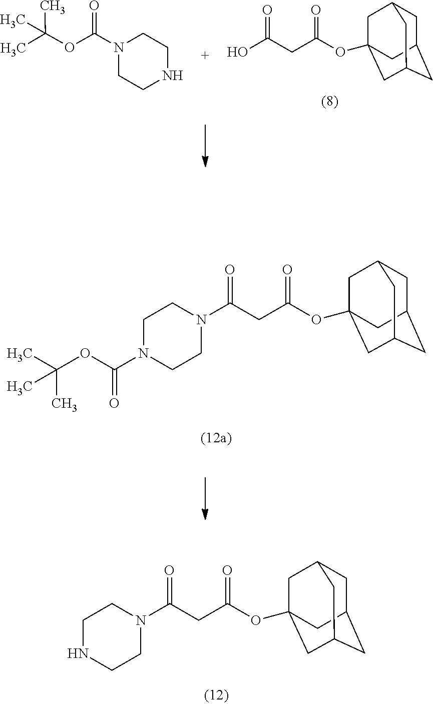 [SMILES: CC(C)(C)OC(=O)N1CCNCC1 ; O=C(O)CC(=O)OC12CC3CC(CC(C3)C1)C2 ; CC(C)(C)OC(=O)N1CCN(C(=O)CC(=O)OC23CC4CC(CC(C4)C2)C3)CC1 ; O=C(CC(=O)N1CCNCC1)OC12CC3CC(CC(C3)C1)C2]

The synthesis was carried out analogously to Synthesis Example 4 from malonic acid adamantyl ester (8) (150 mg, 0.630 mmol), 1-piperazine carbamic acid tert-butyl ester (117 mg, 0.630 mmol) and TEA (176 μl, 1.26 mmol) in DMF (7 ml) with 512 μl of a 1.6 M solution of T3P in ethyl acetate. Purification of the residue by column chromatography (dichloromethane/ethyl acetate 90:10→50:50) yielded the Boc-protected title compound (12a) in the form of a white solid (232 mg).

MS (ESI+): m/z (%) 407 (100, [M+H]⁺), 429 (100, [M+Na]⁺), 836 (100, [2M+Na]⁺).

The product thus obtained (0.571 mmol) was deprotected analogously to Synthesis Example 4, yielding the hydrochloride of the title compound (12) in the form of a white solid (169 mg).

¹H NMR (DMSO-d₆): δ=1.6 (t, 6H, J=3.0 Hz), 2.03 (d, 6H, J=3.0 Hz), 2.11 (brs, 3H), 3.05 (dt, 4H, J=22.6, 5.2 Hz), 3.32 (brs, 1H), 3.48 (s, 2H), 3.63 (dt, 4H, J=22.6, 5.2 Hz) MS (ESI+): m/z (%) 307 (100, [M+H]⁺), 329 (25, [M+Na]⁺), 635 (15, [2M+Na]⁺).

### Synthesis Example 6

### Preparation of Malonic Acid adamantan-1-yl ester 4-(4-aminobutyryl)piperazine Amide (13)

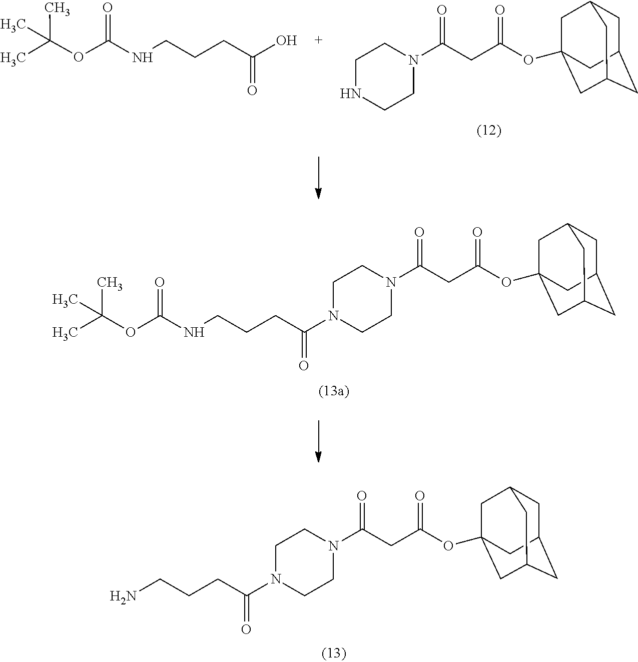 [SMILES: CC(C)(C)OC(=O)NCCCC(=O)O ; O=C(CC(=O)N1CCNCC1)OC12CC3CC(CC(C3)C1)C2 ; CC(C)(C)OC(=O)NCCCC(=O)N1CCN(C(=O)CC(=O)OC23CC4CC(CC(C4)C2)C3)CC1 ; NCCCC(=O)N1CCN(C(=O)CC(=O)OC23CC4CC(CC(C4)C2)C3)CC1]

The synthesis was carried out analogously to Synthesis Example 4 from malonic acid adamantan-1-yl ester piperazine amide (12) (180 mg, 0.525 mmol), 4-(tert-butoxycarbonylamino)butanoic acid (107 mg, 0,525 mmol) and triethylamine (TEA) (219 μl, 1.575 mmol) in DMF (7 ml) with 427 μl of a 1.6 M solution of T3P in ethyl acetate. Purification by column chromatography (dichloromethane/ethyl acetate 80:20) yielded the Boc-protected title compound (13a) in the form of a white solid (190 mg).

MS (ESI+): m/z (%) 492 (100, [M+H]⁺), 514 (100, [M+Na]⁺), 392 (72, {M+H−Boc]⁺).

The product thus obtained (0.386 mmol) was deprotected analogously to Synthesis Example 4, yielding the hydrochloride of the title compound (12) in the form of a white powder (151 mg).

¹H NMR (DMSO-d₆): 1.59 (brs, 6H), 1.76 (quint, 2H, J=7.4 Hz), 2.03 (d, 6H, J=3 Hz), 2.10 (brs, 3H), 2.45 (quint, 2H, J=7.4 Hz), 2.74-2.83 (m, 2H), 3.34 (brs, 2H), 3.40-3.45 (m, 8H), 3.54 (s, 2H). MS (ESI+): m/z (%) 392 (100, [M+H]⁺), 414 (100, [M+Na]⁺), 430 (10, [M+K]⁺), 805 (69, [2M+Na]⁺).

### Synthesis Example 7

### Preparation of Malonic Acid adamantan-1-yl Ester 4-aminopiperidine Amide (14)

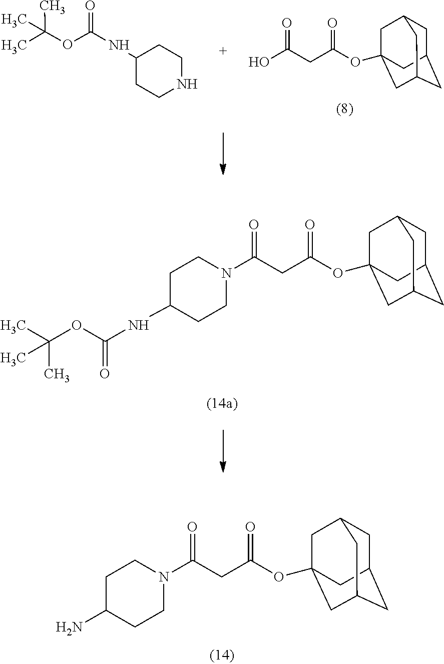 [SMILES: CC(C)(C)OC(=O)NC1CCNCC1 ; O=C(O)CC(=O)OC12CC3CC(CC(C3)C1)C2 ; CC(C)(C)OC(=O)NC1CCN(C(=O)CC(=O)OC23CC4CC(CC(C4)C2)C3)CC1 ; NC1CCN(C(=O)CC(=O)OC23CC4CC(CC(C4)C2)C3)CC1]

The synthesis was carried out analogously to Synthesis Example 4 from malonic acid adamantan-1-yl ester (8) (150 mg, 0,630 mmol), piperidine-4-yl-carbamic acid tertbutyl ester (126 mg, 0,630 mmol) and triethylamine (TEA) (176 μl, 1.26 mmol) in DMF (7 ml) with 512 μl of a 1.6 M solution of T3P in ethyl acetate. Purification by column chromatography (dichloromethane/ethyl acetate 90:10→80:20) yielded the Boc-protected title compound (14a) in the form of a white solid (236 mg).

MS (ESI+): m/z (%) 421 (100, [M+H]⁺), 443 (100, [M+Na]⁺), 864 (100, [2M+Na]⁺).

The product thus obtained (0,561 mmol) was deprotected analogously to Synthesis Example 4, yielding the hydrochloride of the title compound (14) in the form of a white powder (175 mg).

MS (ESI+): m/z (%) 321 (100, [M+H]⁺), 343 (7, [M+Na]⁺), 641 (24, [2M+H]⁺), 663 (95, [2M+Na]⁺).

### Synthesis Example 8

### Preparation of Malonic Acid adamantan-1-yl Ester (4-(4-aminobutyryl)amino)piperidine Amide (15)

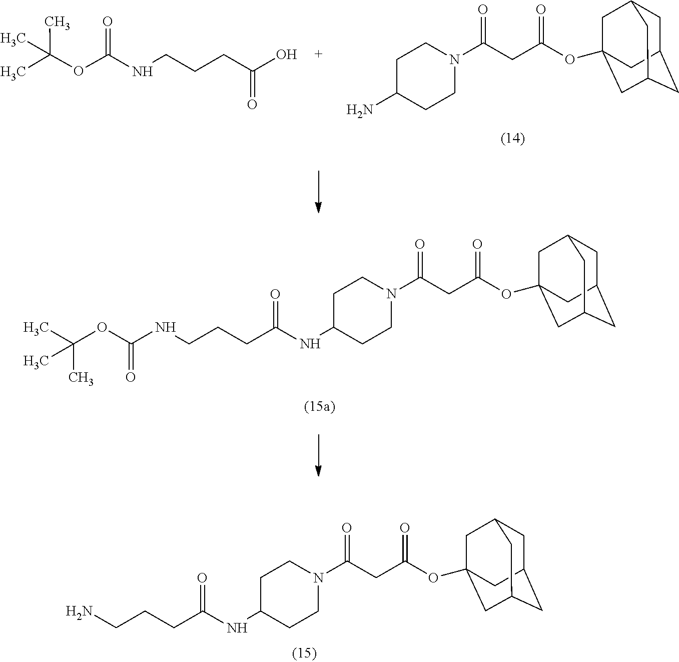 [SMILES: CC(C)(C)OC(=O)NCCCC(=O)O ; NC1CCN(C(=O)CC(=O)OC23CC4CC(CC(C4)C2)C3)CC1 ; CC(C)(C)OC(=O)NCCCC(=O)NC1CCN(C(=O)CC(=O)OC23CC4CC(CC(C4)C2)C3)CC1 ; NCCCC(=O)NC1CCN(C(=O)CC(=O)OC23CC4CC(CC(C4)C2)C3)CC1]

The synthesis was carried out analogously to Synthesis Example 4 from malonic acid adamantan-1-yl ester 4-aminopiperidine amide (14) (140 mg, 0.392 mmol), 4-(tert-butoxycarbonylamino)butanoic acid (80 mg, 0.392 mmol) and triethylamine (TEA) (164 μl, 1.176 mmol) in DMF (7 ml) with 319 μl of a 1.6 M Solution of T3P in ethyl acetate. Purification by column chromatography (dichloromethane/ethyl acetate 80:20→ethyl acetate) yielded the Boc-protected title compound (15a) in the form of a yellow oil (83 mg).

MS (ESI+): m/z (%) 506 (100, [M+H]⁺), 528 (100, [M+Na]⁺), 406 (78, [M-Boc+H]⁺).

The product thus obtained (0.164 mmol) was deprotected analogously to Synthesis Example 4, yielding the hydrochloride of the title compound (14) in the form of a yellow powder (80 mg).

¹H NMR (DMSO-d₆): δ=1.64 (dt, 3H, J=13.1, 2.8 Hz), 1.66 (dt, 3H, J=13.1, 2.8 Hz), 1.74 (sept, 3H, J=2.8 Hz), 1.80-1.87 (m, 6H), 2.09-2.13 (m, 6H), 2.16 (t, 2H, J=7.4 Hz), 2.65 (t, 2H, J=7.7 Hz), 3.04-3.13 (m, 2H), 3.35 (ddd, 2H, J=14.7, 6.8, 2.8 Hz), 3.55 (s, 2H), 4.11-4.21 (m, 1H), 7.97 (brs, 2H). MS (ESI+): m/z (%) 406 (100, [M+H]⁺), 428 (100, [M+Na]⁺), 811 (100, [2M+H]⁺), 833 (53, [2M+Na]⁺).

### Synthesis Example 9

### Preparation of Malonic Acid Methyl Ester N-(adamantan-1-yl)amide (16)

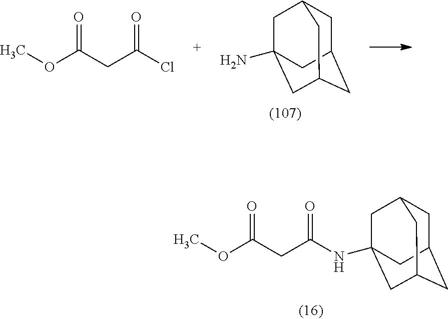

Malonic acid methyl ester chloride (322 μl, 3.005 mmol) was dissolved in 30 ml of dichloromethane under an inert gas atmosphere and cooled to −20° C., after which a solution of 1-adamantanamine (107) (1.0 g, 6.612 mmol) in 10 ml of dichloromethane was slowly added using a syringe. The reaction mixture was stirred at room temperature for 1 h, quenched with a saturated aqueous NH₄Cl solution and extracted with dichloromethane. The organic phase was dried over MgSO₄, filtered and evaporated to dryness, yielding the title compound (760 mg) in the form of white crystals.

¹H NMR (CDCl₃): δ=1.68 (m, 6H), 2.01-2.10 (m, 9H), 3.22 (s, 2H), 3.74 (s, 3H), 6.64 (brs, 1H). MS (ESI+): m/z (%) 252 (100, [M+H]⁺), 274 (100, [M+Na]⁺), 503 (78, [2M+H]⁺).

### Synthesis Example 10

### Preparation of N-(adamantan-1-yl)malonic Acid Amide (17)

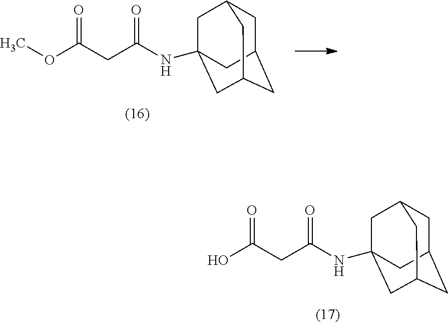 [SMILES: COC(=O)CC(=O)NC12CC3CC(CC(C3)C1)C2 ; O=C(O)CC(=O)NC12CC3CC(CC(C3)C1)C2]

To a solution of 750 mg (2.98 mmol) of malonic acid methyl ester N-(adamantan-1-yl)amide (16) in 30 ml of THF/H₂O (2:1), 6 ml of 1 M aqueous NaOH was added and stirred at room temperature. After 1 h, the THF was removed on a rotary evaporator, and the remaining aqueous solution was acidified with 2 N HCl and extracted with ethyl acetate. The organic phase was dried over MgSO₄, filtered and evaporated to dryness, yielding the title compound (17) as an off-white powder (670 mg).

MS (ESI−): m/z (%) 236 (100, [M−H]⁻), 473 (100, [2M−H]⁻).

### Synthesis Example 11

### Preparation of N-adamantan-1-yl-N′-(3-aminopropyl)malonic Acid Diamide (18)

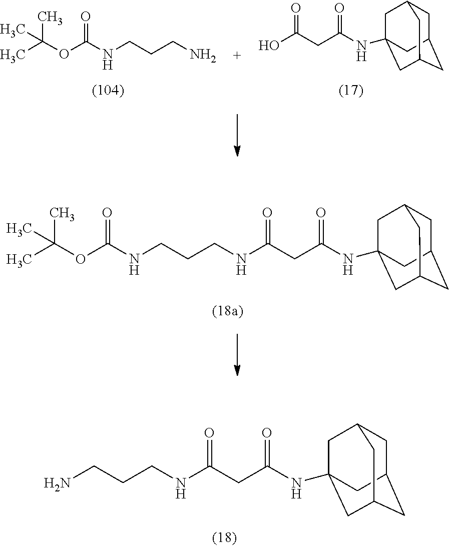 [SMILES: CC(C)(C)OC(=O)NCCCN ; O=C(O)CC(=O)NC12CC3CC(CC(C3)C1)C2 ; CC(C)(C)OC(=O)NCCCNC(=O)CC(=O)NC12CC3CC(CC(C3)C1)C2 ; NCCCNC(=O)CC(=O)NC12CC3CC(CC(C3)C1)C2]

To a solution of N-(adamantan-1-yl)malonic acid amide (17) (50 mg, 0.211 mmol), 3-aminopropyl carbamic acid tert-butyl ester (104) (37 mg, 0.211 mmol) and 4-dimethylaminopyridine (DMAP) (13 mg, 0.106 mmol) in dichloromethane (8 ml), diisopropylcarbodiimide (DIC) (40 μl, 0.253 mmol) was added. After stirring for 4 h while allowing to warm to room temperature, the reaction mixture was evaporated to dryness. The residue containing the Boc-protected N-adamantan-1-yl-N′-(3-aminopropyl)malonic acid diamide (18a) (59 mg, 0.202 mmol) was used in the next step without further purification.

MS (ESI+): m/z (%) 394 (100, [M+H]⁺), 415 (100, [M+Na]⁺), 294 (70, [M-BOC+H]⁺), 787 (40, [2M+H]⁺).

The residue thus obtained was dissolved in 6 ml of dichloromethane, 2 ml of 4 N HCl in dioxane were added, and the mixture was stirred for 2 h at room temperature. The volatiles were then removed in vacuo, yielding the hydrochloride of the title compound (18) in the form of a yellow oil (55 mg).

¹H NMR (DMSO-d₆): δ=1.61 (m, 6H, adamantyl), 1.68 (quint, 2H, J=7.1 Hz), 1.90-2.00 (m, 9H, adamantyl), 2.74-2.82 (m, 2H), 2.98 (s, 2H), 3.12 (q, 2H, J=6.42 Hz), 7.58 (s, 1H). MS (ESI+): m/z (%) 294 (90, [M+H]⁺), 316 (100, [M+Na]⁺), 608 (45, [2M+Na]⁺).

### Synthesis Example 12

### Preparation of N-adamantan-1-yl-N′-(3-aminopropyl)-N′-methyl Malonic Acid Diamide (19)

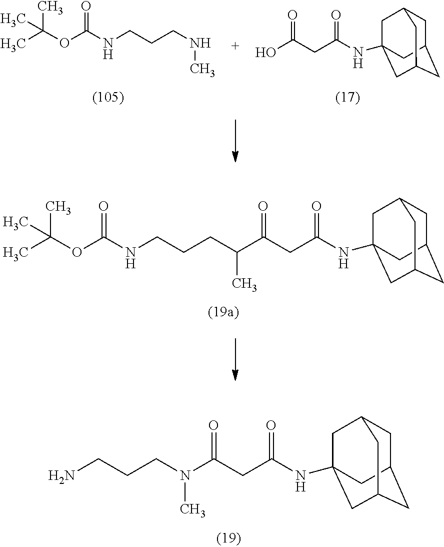 [SMILES: CNCCCNC(=O)OC(C)(C)C ; O=C(O)CC(=O)NC12CC3CC(CC(C3)C1)C2 ; CC(CCCNC(=O)OC(C)(C)C)C(=O)CC(=O)NC12CC3CC(CC(C3)C1)C2 ; CN(CCCN)C(=O)CC(=O)NC12CC3CC(CC(C3)C1)C2]

The synthesis was carried out analogously to Synthesis Example 4 from N-(adamantan-1-yl)malonic acid amide (17) (50 mg, 0.211 mmol), 3-(methylamino)propyl carbamic acid tert-butyl ester (105) (40 mg, 0.211 mmol), and triethylamine (TEA) (60 μl, 0.422 mmol) in DMF (10 ml) with 175 μl of a 1.6 M solution of T3P in ethyl acetate. Purification by column chromatography (dichloromethane/ethyl acetate 70:30) yielded the Boc-protected title compound (19a) in the form of a yellow oil (83 mg).

MS (ESI+): m/z (%) 408 (100, [M+H]⁺), 430 (100, [M+Na]⁺), 308 (100, [M-BOC+H]⁺), 815 (50, [2M+H]⁺).

The product thus obtained (46 mg, 0.113 mmol) was deprotected analogously to Synthesis Example 4, yielding the hydrochloride of the title compound (19) in the form of a yellow oil (33 mg).

¹H NMR (DMSO-d₆): δ=1.61 (m, 6H, adamantyl), 1.74 (quint, 2H, J=5.5 Hz), 1.97-2.07 (m, 9H, adamantyl), 2.75-2.84 (m, 2H), 2.93 (s, 3H), 3.26 (s, 2H), 3.35 (t, 2H, J=5.5 Hz), 3.41 (brs, 2H), 7.50 (s, 1H). MS (ESI+): m/z (%) 308 (100, [M+H]⁺), 330 (33, [M+Na]⁺), 615 (100, [2M+H]⁺).

### Synthesis Example 13

### Preparation of N-adamantan-1-yl-N′-piperazine Malonic Acid Diamide (20)

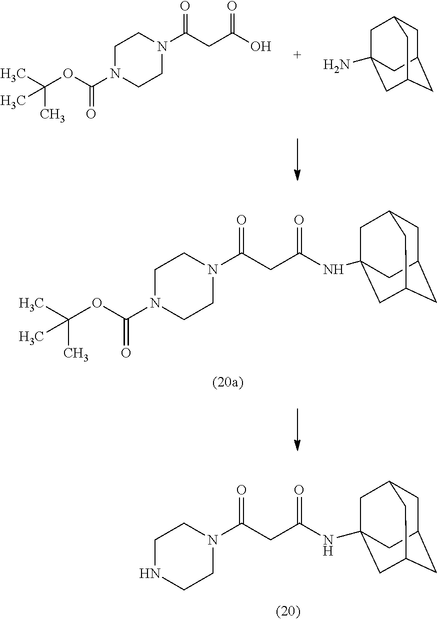 [SMILES: CC(C)(C)OC(=O)N1CCN(C(=O)CC(=O)O)CC1 ; NC12CC3CC(CC(C3)C1)C2 ; CC(C)(C)OC(=O)N1CCN(C(=O)CC(=O)NC23CC4CC(CC(C4)C2)C3)CC1 ; O=C(CC(=O)N1CCNCC1)NC12CC3CC(CC(C3)C1)C2]

The synthesis was carried out analogously to Synthesis Example 4 from commercial malonic acid 4-(tert-butoxycarbonyl)piperidine amide (250 mg, 0.918 mmol), 1-adamantanamine (107) (139 mg, 0.918 mmol), and triethylamine (TEA) (265 μl, 1.84 mmol) in DMF (17 ml) with 750 μl of a 1.6 M solution of T3P in ethyl acetate. Purification by column chromatography (dichloromethane/ethyl acetate 90:10→50:50) yielded the Boc-protected title compound (20a) in the form of a white solid (260 mg).

¹H NMR (CDCl₃): δ=1.46 (s, 9H), 1.67 (t, 7H, J=3.9 Hz), 1.98 (d, 6H, J=3.1 Hz), 2.06 (brs, 3H), 3.24 (s, 2H), 3.40-3.46 (m, 4H), 3.52-3.55 (m, 2H), 3.58-3.61 (m, 2H), 6.73 (brs, 1H). MS (ESI+): m/z (%) 406 (100, [M+H]⁺), 428 (100, [M+Na]⁺), 833 (2M+Na]⁺). The product thus obtained (0.647 mmol) was deprotected analogously to Synthesis Example 4, yielding the hydrochloride of the title compound (20) in the form of a white powder (192 mg).

¹H NMR (DMSO-d₆): 1.61 (brs, 6H), 1.9 (d, 6H, J=2.95 Hz), 2.00 (brs, 3H), 3.00 (brs, 2H), 3.10 (brs, 2H), 3.66-3.68 (m, 4H), 4.01 (brs, 2H). MS (ESI+): m/z (%) 306 (100, [M+H]⁺), 329 (7, [M+Na]⁺), 633 (95, [2M+Na]⁺).

### Synthesis Example 14

### Preparation of N-adamantan-1-yl-N′-(4-(4-aminobutyryl)piperazine)malonic Acid Diamide (21)

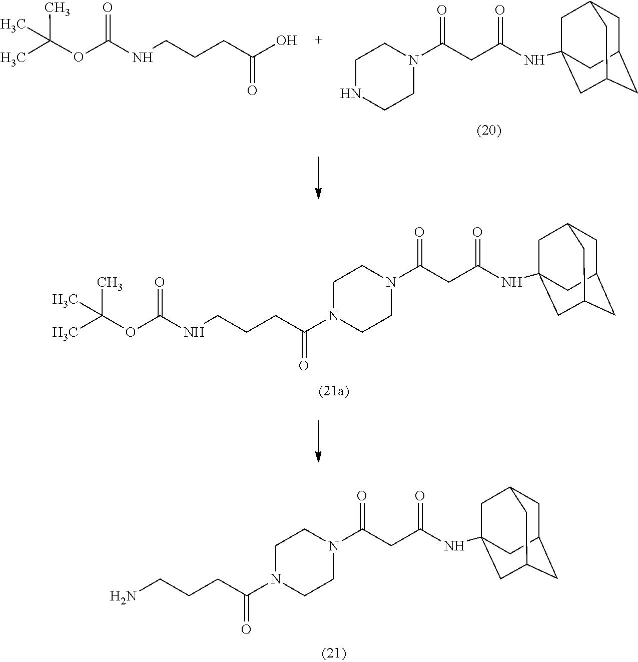 [SMILES: CC(C)(C)OC(=O)NCCCC(=O)O ; O=C(CC(=O)N1CCNCC1)NC12CC3CC(CC(C3)C1)C2 ; CC(C)(C)OC(=O)NCCCC(=O)N1CCN(C(=O)CC(=O)NC23CC4CC(CC(C4)C2)C3)CC1 ; NCCCC(=O)N1CCN(C(=O)CC(=O)NC23CC4CC(CC(C4)C2)C3)CC1]

The synthesis was carried out analogously to Synthesis Example 4 from the hydrochloride of N-adamantan-1-yl-N′-piperazine malonic acid diamide (20) (140 mg, 0.410 mmol), 4-(tert-butoxycarbonylamino)butanoic acid (83 mg, 0.410 mmol), and triethylamine (TEA) (171 μl, 1.23 mmol) in DMF (7 ml) with 333 μl of a 1.6 M solution of T3P in ethyl acetate. Purification by column chromatography (ethyl acetate/methanol 95:5) yielded the Boc-protected title compound (21a) in the form of a white solid (180 mg).

MS (ESI+): m/z (%) 491 (100, [M+H]⁺), 513 (100, [M+Na]⁺), 391 (38, [M-Boc+H]⁺).

The product thus obtained (0.367 mmol) was deprotected analogously to Synthesis Example 4, yielding the hydrochloride of the title compound (21) in the form of a white powder (150 mg).

¹H NMR (DMSO-d₆): 1.59 (brs, 6H), 1.77 (quint, 2H, J=6.4 Hz), 1.89 (brs, 6H), 1.98 (brs, 3H), 2.43-2.47 (m, 2H), 2.76-2.81 (m, 2H), 3.26-3.30 (m, 2H), 3.39-3.45 (m, 8H), 4.34 (brs, 2H), 7.61 (s, 1H).

### Synthesis Example 15

### Preparation of N-(3-aminopropyl)-N′-morpholine Malonic Acid Diamide (22)

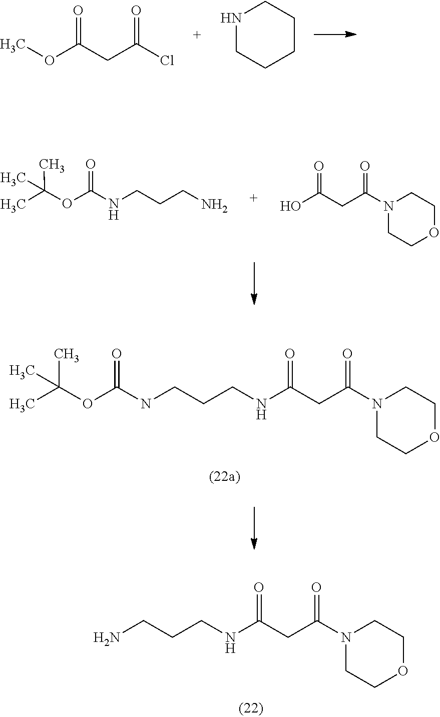

The synthesis was carried out analogously to Synthesis Example 4 from commercial N-morpholinomalonic acid amide (90 mg, 0.520 mmol), 3-aminopropyl carbamic acid tert-butyl ester (N-Boc-1,3-propane diamine) (104) (91 mg, 0.520 mmol), and triethylamine (TEA) (145 μl, 1.04 mmol) in DMF (5 ml) with 423 μl of a 1.6 M solution of T3P in ethyl acetate. Purification by column chromatography (ethyl acetate/methanol 95:5) yielded the Boc-protected title compound (22a) in the form of a yellow oil (90 mg).

MS (ESI+): m/z (%) 330 (100, [M+H]⁺), 352 (100, [M+Na]⁺), 230 (100, [M-Boc+H]⁺), 681 (100, [2M+Na]⁺).

The product thus obtained (0.273 mmol) was deprotected analogously to Synthesis Example 4, yielding the hydrochloride of the title compound (22) in the form of a yellowish oil (76 mg).

MS (ESI+): m/z (%) 230 (100, [M+H]⁺), 252 (100, [M+Na]⁺), 459 (11, [2M+H]⁺), 481 (100, [2M+Na]⁺).

### Synthesis Example 16

### Preparation of N-(3-aminopropyl)-N′-piperidine Malonic Acid Diamide (23)

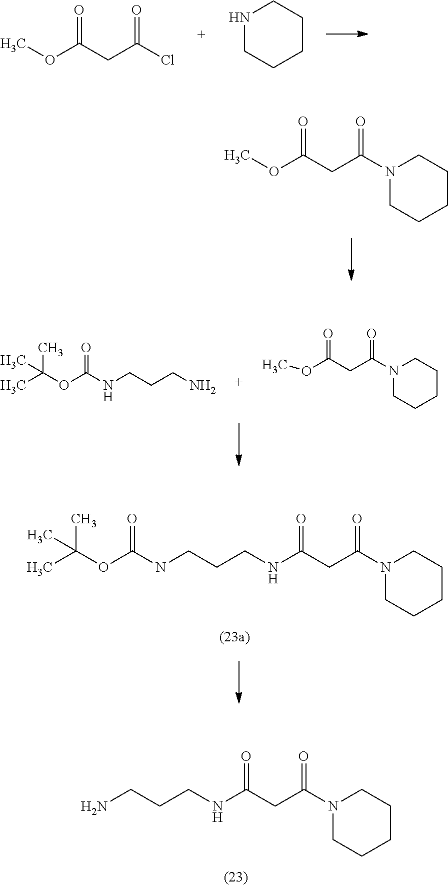

The synthesis was carried out analogously to a combination of Synthesis Examples 9, 10 and 4, wherein first, in analogy to Synthesis Example 9, malonic acid methyl ester N-piperidine amide was prepared from malonic acid methyl ester chloride (250 μl, 2.33 mmol) and piperidine (506 μl, 5.126 mmol) at −20° C. in 10 ml of dichloromethane, which was then hydrolyzed in THF/H₂O (2:1) with aqueous NaOH in analogy to Synthesis Example 10 to yield malonic acid N-piperidine amide.

MS (ESI+): m/z (%) 172 (100, [M+H]⁺), 194 (50, [M+H]⁺).

This (280 mg, 1.636 mmol) was then, in analogy to Synthesis Example 4, reacted with 3-aminopropyl carbamic acid tert-butyl ester (N-Boc-1,3-propane diamine) (285 mg, 1.636 mmol) and TEA (456 μl, 3.272 mmol) in DMF (6 ml) with 1.33 ml of a 1.6 M solution of T3P in ethyl acetate. Purification by column chromatography (dichloromethane/ethyl acetate 50:50→ethyl acetate) yielded the Boc-protected title compound (23a) in the form of a colorless oil (370 mg).

¹H NMR (CDCl₃): δ=1.43 (s, 9H), 1.53-1.60 (m, 4H), 1.63-1.68 (m, 5H), 3.14 (q, 2H, J=6.5 Hz), 3.31 (s, 2H), 3.33 (q, 2H, J=6.5 Hz), 3.45 (t, 2H, J=4.8 Hz), 3.56 (t, 2H, J=4.8 Hz), 7.78 (s, 1H). MS (ESI+): m/z (%) 328 (100, [M+H]⁺), 350 (100, [M+Na]⁺), 228 (80, [M-Boc+H]⁺), 677 (100, [2M+Na]⁺).

The product thus obtained (1.13 mmol) was deprotected analogously to Synthesis Example 4, yielding the hydrochloride of the title compound (23) in the form of a white powder (330 mg).

¹H NMR (DMSO-d₆): δ=1.37-1.43 (m, 2H), 1.45-1.50 (m, 2H), 1.53-1.59 (m, 2H), 1.69 (quint, 2H, J=8.3 Hz), 2.75-2.83 (m, 2H), 3.12 (q, 2H, 4.8 Hz), 3.35-3.41 (m, 4H), 7.96 (s, 1H). MS (ESI+): m/z (%) 228 (100, [M+H]⁺), 250 (16, [M+Na]⁺), 455 (40, [2M+H]⁺).

### Synthesis Example 17

### Preparation of N-(3-aminopropyl)-N′-decahydroquinoline Malonic Acid Diamide (24)

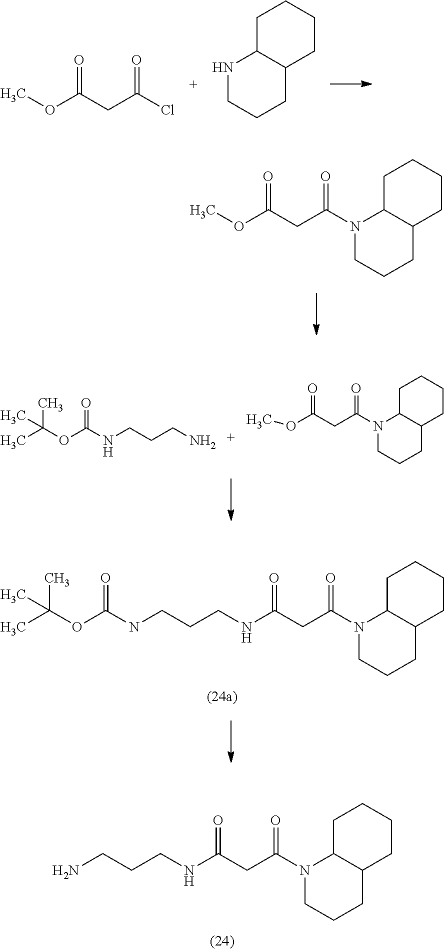

The synthesis was carried out analogously to Synthesis Example 16 from malonic acid methyl ester chloride (175 μl, 1.63 mmol) and trans-decahydroquinoline (500 mg, 3.59 mmol) to malonic acid methyl ester N-decahydroquinoline amide, which was hydrolyzed to yield malonic acid N-decahydroquinoline amide.

MS (ESI+): m/z (%) 226 (100, [M+H]⁺), 248 (85, [M+Na]⁺).

This (190 mg, 0.843 mmol) was reacted with 3-aminopropyl carbamic acid tert-butyl ester (N-Boc-1,3-propane diamine) (147 mg, 0.843 mmol) and TEA (236 μl, 1.686 mmol) in DMF (6 ml) with 685 μl of a 1.6 M solution of T3P in ethyl acetate. Purification by column chromatography (dichloromethane/ethyl acetate 50:50→40:60) yielded the Boc-protected title compound (24a) in the form of a colorless oil (230 mg).

MS (ESI+): m/z (%) 382 (100, [M+H]⁺), 404 (80, [M+Na]⁺), 282 (37, [M-Boc+H]⁺), 785 (15, [2M+Na]⁺).

The product thus obtained (0.603 mmol) was deprotected in an analogous manner, yielding the hydrochloride of the title compound (24) in the form of a white powder (215 mg).

MS (ESI+): m/z (%) 282 (100, [M+H)⁺], 304 (30, [M+Na]⁺), 563 (80, [2M+H]⁺), 585 (60, [2M+Na]⁺).

### Synthesis Example 18

### Preparation of N-(3-aminopropyl)-N′-decahydroisoquinoline Malonic Acid Diamide (25)

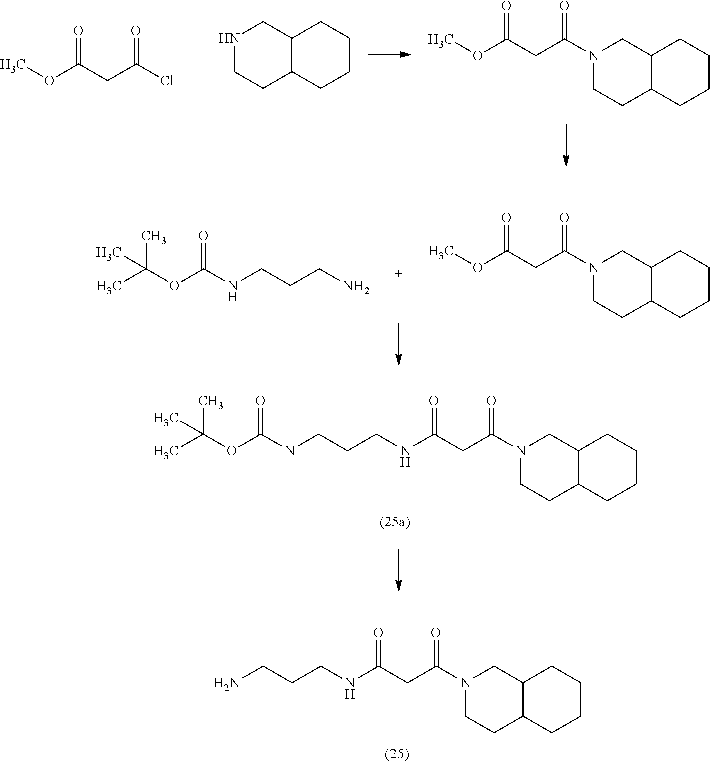

The synthesis was carried out analogously to Synthesis Example 17 from malonic acid methyl ester chloride (175 μl, 1.63 mmol) and decahydroisoquinoline (500 mg, 3.59 mmol) to malonic acid methyl ester N-decahydroisoquinoline amide, which was hydrolyzed to yield malonic acid N-decahydroisoquinoline amide.

MS (ESI+): m/z (%) 226 (100, [M+H]⁺), 248 (100, [M+Na]⁺), 473 (36, [2M+Na]⁺).

This (272 mg, 1.207 mmol) was reacted with 3-aminopropyl carbamic acid tert-butyl ester (N-Boc-1,3-propane diamine) (210 mg, 1.207 mmol) and TEA (336 μl, 2.414 mmol) in DMF (10 ml) with 980 μl of a 1.6 M solution of T3P in ethyl acetate. Purification by column chromatography (ethyl acetate) yielded the Boc-protected title compound (25a) in the form of a colorless oil (306 mg).

MS (ESI+): m/z (%) 382 (100, [M+H]⁺), 404 (100, [M+Na]⁺), 282 (100, [M-Boc+H]⁺), 785 (100, [2M+Na]⁺).

The product thus obtained (0.786 mmol) was deprotected in an analogous manner, yielding the hydrochloride of the title compound (25) in the form of a white powder (280 mg).

MS (ESI+): m/z (%) 282 (100, [M+H]+), 304 (20, [M+Na]⁺), 563 (40, [2M+H]⁺), 585 (50, [2M+Na]⁺).

### Synthesis Example 19

### Preparation of N-(3-aminopropyl)-N′-(pinane-10-yl)malonic Acid Diamide (26)

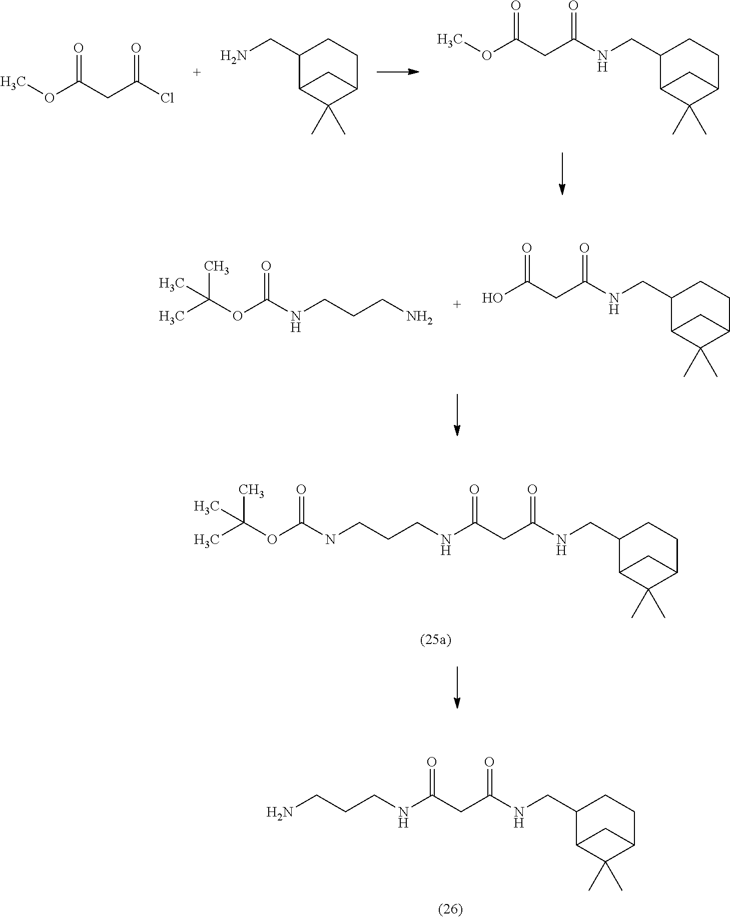

The synthesis was carried out analogously to Synthesis Example 16 from malonic acid methyl ester chloride (250 μl, 2.33 mmol) and (−)-cis-myrtanylamin (10-pinanamine) (860 mg, 5.13 mmol) to malonic acid methyl ester N-(pinane-10-yl)amide, which was hydrolyzed to yield malonic acid N-(pinane-10-yl)amide.

MS (ESI+): m/z (%) 240 (100, [M+H]⁺), 262 (55, [M+Na]⁺), 501 (10, [2M+Na]⁺). MS (ESI−) m/z (%) 238 (100, [M−H]⁻), 477 (100, [2M−H]⁻).

This (295 mg, 1.327 mmol) was reacted with 3-aminopropyl carbamic acid tert-butyl ester (N-Boc-1,3-propane diamine) (231 mg, 1.327 mmol) and TEA (371 μl, 2.654 mmol) in DMF (7 ml) with 1.1 ml of a 1.6 M solution of T3P in ethyl acetate. Purification by column chromatography (dichloromethane/ethyl acetate 50:50→ethyl acetate) yielded the Boc-protected title compound (26a) in the form of a colorless oil (278 mg).

¹H NMR (CDCl₃): δ=0.88 (d, 1H, J=8.5 Hz), 1.02 (s, 3H), 1.18 (s, 3H), 1.44 (s, 9H), 1.45-1.50 (m, 1H), 1.59-1.67 (m, 4H), 1.84-1.98 (m, 5H), 2.16-2.24 (m, 1H), 2.33-2.38 (m, 1H), 3.16 (s, 2H), 3.13-3.18 (m, 2H), 3.24-3.34 (m, 4H), 7.05 (s, 1H). MS (ESI+): m/z (%) 396 (100, [M+H]⁺), 296 (100, [M-Boc+H]⁺), 418 (100, [M+Na]+), 791(100, [2M+H]⁺), 813 (100, [2M+Na]⁺).

The product thus obtained (0.703 mmol) was deprotected in an analogous manner, yielding the hydrochloride of the title compound (26) in the form of a white solid (260 mg).

¹H NMR (DMSO-d₆): δ=0.84 (d, 1H, J=8.5 Hz), 0.99 (s, 3H), 1.15 (s, 3H), 1.38-1.45 (m, 1H), 1.65-1.72 (m, 2H), 1.79-1.91 (m, 5H), 2.06-2.14 (m, 1H), 2.29-2.34 (m, 1H), 2.75-2.83 (m, 2H), 3.01 (s, 2H), 3.03-3.08 (m, 2H), 3.10-3.16 (m, 2H), 3.64-3.73 (m, 1H), 7.82 (brs, 2H). MS (ESI+): m/z (%) 296 (100, [M+H]⁺), 318 (90, [M+Na]⁺), 591 (100, [2M+H]⁺), 613 (100, [2M+Na]⁺).

### Synthesis Example 20

### Preparation of N-(3-aminopropyl)-N′-tetrahydroquinoline Malonic Acid Diamide (108)

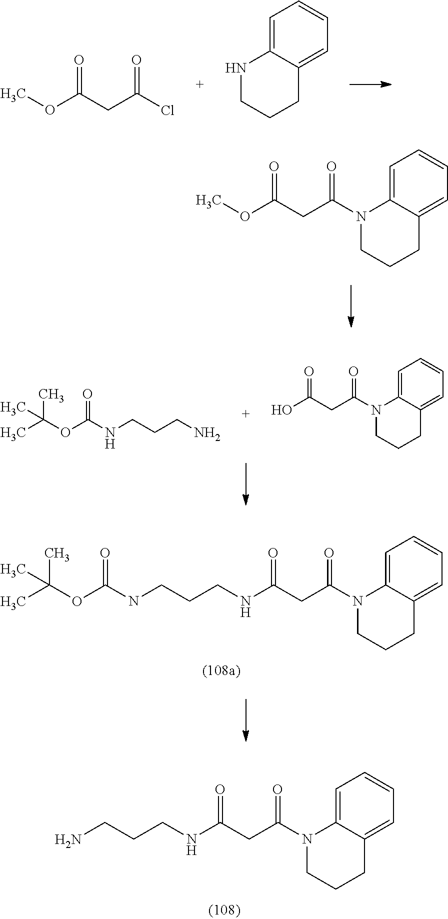

The synthesis was carried out analogously to Synthesis Example 17 from malonic acid methyl ester chloride (175 μl, 1.63 mmol) and 1,2,3,4-tetrahydroquinoline (478 mg, 3.59 mmol) to malonic acid methyl ester N-tetrahydroquinoline amide, which was hydrolyzed to yield malonic acid N-tetrahydroquinoline amide.

MS (ESI+): m/z (%) 220 (100, [M+H]⁺), 242 (100, [M+Na]⁺), 461 (17, [2M+Na]⁺). MS (ESI−) m/z (%) 218 (100, [M−H]⁻).

This (250 mg, 1.140 mmol) was reacted with 3-aminopropyl carbamic acid tert-butyl ester (N-Boc-1,3-propane diamine) (199 mg, 1.140 mmol) and TEA (318 μl, 2.280 mmol) in DMF (10 ml) with 900 μl of a 1.6 M solution of T3P in ethyl acetate. Purification by column chromatography (dichloromethane/ethyl acetate 70:30→40:60) yielded the Boc-protected title compound (108a) in the form of a colorless oil (230 mg).

¹H NMR (CDCl₃): δ=1.43 (s, 9H), 1.67 (quint, 2H, J=6.7 Hz), 1.98 (quint, 2H, J=6.7 Hz), 2.72 (brs, 2H), 3.16 (q, 2H, J=6.2 Hz), 3.34 (q, 2H, J=6.4 Hz), 3.47 (s, 2H), 3.82 (t, 2H, J=6.7 Hz), 5.00 (brs, 1H), 7.09-7.23 (m, 4H). MS (ESI+): m/z (%) 376 (100, [M+H]⁺), 398 (100, [M+Na]⁺), 276 (100,[M-Boc+H]⁺), 773 (100, [2M+Na]⁺). MS (ESI−) m/z (%) 374 (100, [M−H]⁻).

The product thus obtained (275 mg, 0.732 mmol) was deprotected in an analogous manner, yielding the hydrochloride of the title compound (108) in the form of a white powder (264 mg).

¹H NMR (DMSO-d₆): δ=1.67 (quint, 2H, J=7.1 Hz), 1.87 (quint, 2H, J=6.6), 2.69 (t, 2H, J=6.6 Hz), 2.78 (q, 2H, J=6.6 Hz), 3.11 (q, 2H, 6.5 Hz), 3.67 (s, 2H), 3.64-3.72 (m, 2H), 7.09-7.20 (m, 4H), 7.82 (brs, 2H), 8.22 (brs, 1H). MS (ESI+): m/z (%) 276 (100, [M+H]⁺), 298 (8, [M+Na]⁺), 551 (11, [2M+H]⁺), 573 (18, [2M+Na]⁺).

### Synthesis Example 21

### Preparation of Malonic Acid adamantan-1-ylester-N-(3-dimethylaminopropyl)amide (27)

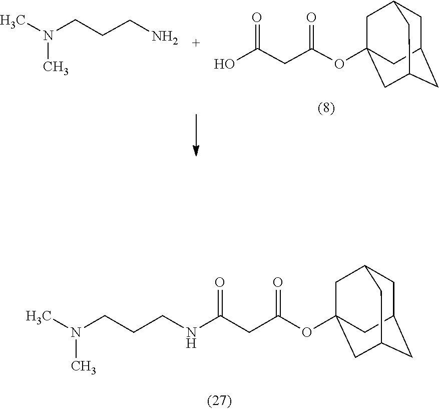

To a solution of malonic acid adamantan-1-yl ester (8) (350 mg, 1.469 mmol) and 3-aminopropyl dimethylamine (150 mg, 1.469 mmol) in DMF (10 ml), triethylamine (TEA) (409 μl, 2.398 mmol) and 1.2 ml of a 1.6 M solution of propane phosphonic acid anhydride (T3P) in ethyl acetate were slowly added and stirred for 1 h at room temperature. The reaction mixture was then mixed with saturated saline for hydrolysis and extracted with ethyl acetate. The extract was dried over MgSO₄, filtered and evaporated to dryness. The residue was purified by column chromatography (dichloromethane/ethyl acetate 50:50→ethyl acetate/methanol 70:30), yielding the title compound (27) in the form of a colorless oil (400 mg).

¹H NMR (CDCl₃): δ=1.66 (m, 6H, adamantyl), 1.72 (quint, 2H, J=6.77 Hz), 2.04-2.11 (m, 9H, adamantyl), 2.28 (s, 6H), 2.42 (t, 2H, J=6.92 Hz), 3.19 (s, 2H), 3.45 (q, 2H, J=6.30 Hz), 7.62 (brs, 1H). MS (ESI+): m/z (%) 323 (100, [M+H]⁺), 345 (11, [M+Na]⁺).

### Synthesis Example 22

### Preparation of Malonic Acid adamantan-1-yl ester-N-(3-morpholinopropyl)amide (28)

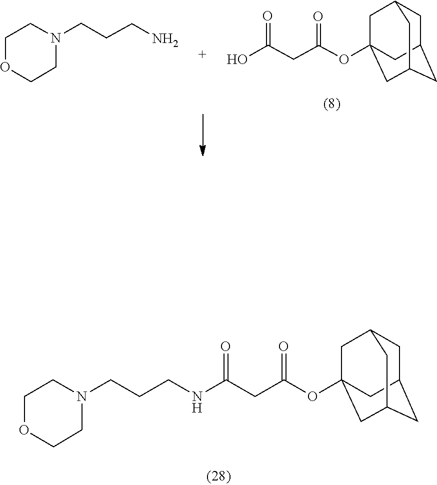 [SMILES: NCCCN1CCOCC1 ; O=C(O)CC(=O)OC12CC3CC(CC(C3)C1)C2 ; O=C(CC(=O)OC12CC3CC(CC(C3)C1)C2)NCCCN1CCOCC1]

The synthesis was carried out analogously to Synthesis Example 21 from malonic acid adamantan-1-yl ester (8) (190 mg, 0.797 mmol), 3-morpholinopropylamine (115 mg, 0.797 mmol) and triethylamine (TEA) (222 μl, 1.594 mmol) in DMF (5 ml) with 650 μl of a 1.6 M solution of T3P in ethyl acetate. Purification by column chromatography (ethyl acetate→ethyl acetate/methanol 90:10) yielded the title compound (28) in the form of a colorless oil (250 mg).

¹H NMR (CDCl₃): δ=1.65-1.74 (m, 8H), 2.10 (m, 6H, adamantyl), 2.17 (brs, 3H, adamantyl), 2.40-2.44 (m, 6H), 3.19 (s, 2H), 3.33-3.38 (m, 2H), 3.72 (t, 4H, J=4.68 Hz), 7.61 (brs, 1H). MS (ESI+): m/z (%) 365 (100, [M+H]⁺), 387 (20, [M+Na]⁺), 751 (67, [2M+Na]⁺).

### Synthesis Example 23

### Preparation of Malonic Acid adamantan-1-yl ester 4-(3-trifluoromethylbenzyl)piperazine amide (29)

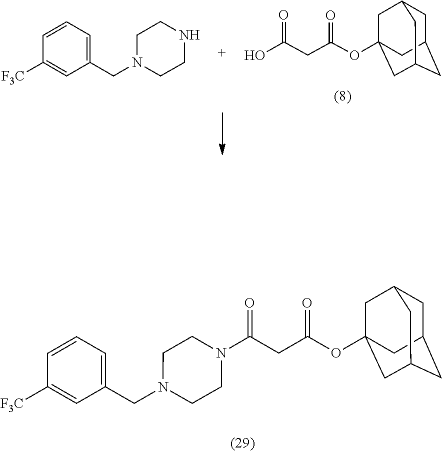 [SMILES: FC(F)(F)c1cccc(CN2CCNCC2)c1 ; O=C(O)CC(=O)OC12CC3CC(CC(C3)C1)C2 ; O=C(CC(=O)N1CCN(Cc2cccc(C(F)(F)F)c2)CC1)OC12CC3CC(CC(C3)C1)C2]

The synthesis was carried out analogously to Synthesis Example 21 from malonic acid adamantan-1-yl ester (8) (85 mg, 0.357 mmol), 1-(3-trifluoromethyl benzyl)piperazine (87 mg, 0.357 mmol) and triethylamine (TEA) (100 μl, 0.714 mmol) in DMF (2 ml) with 290 μl of a 1.6 M solution of T3P in ethyl acetate. Purification by column chromatography (dichloromethane/ethyl acetate 90:10→50:50) yielded the title compound (29) in the form of a colorless oil (116 mg).

¹H NMR (CDCl₃): δ=1.65 (m, 6H, adamantyl), 2.10-2.17 (m, 9H, adamantyl), 2.45 (q, 4H, J=4.61 Hz), 3.37 (s, 2H), 3.44 (t, 2H, J=5.05 Hz), 3.57 (s, 2H), 3.66 (t, 2H, J=5.05 Hz), 7.42-7.59 (m, 4H, H-Aryl). MS (ESI+): m/z (%) 465 (100, [M+H]⁺), 487 (42, [M+Na]⁺), 951 (7, [2M+Na]⁺).

### Synthesis Example 24

### Preparation of Malonic Acid adamantan-1-yl ester 4-(3-fluorobenzyl)piperazine Amide (30)

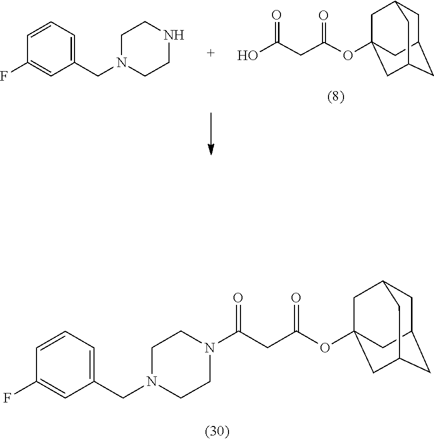 [SMILES: Fc1cccc(CN2CCNCC2)c1 ; O=C(O)CC(=O)OC12CC3CC(CC(C3)C1)C2 ; O=C(CC(=O)N1CCN(Cc2cccc(F)c2)CC1)OC12CC3CC(CC(C3)C1)C2]

The synthesis was carried out analogously to Synthesis Example 21 from malonic acid adamantan-1-ylester (8) (127 mg, 0.533 mmol), 1-(3-fluorobenzyl)piperazine (104 mg, 0.533 mmol) and triethylamine (TEA) (149 μl, 1.07 mmol) in DMF (4 ml) with 433 μl of a 1.6 M solution of T3P in ethyl acetate. Purification by column chromatography (dichloromethane/ethyl acetate 80:20→50:50) yielded the title compound (30) in the form of a colorless oil (172 mg).

¹H NMR (CDCl₃): δ=1.65 (m, 6H, adamantyl), 2.10-2.16 (m, 9H, adamantyl), 2.44 (q, 4H, J=5.20 Hz), 3.37 (s, 2H), 3.43 (t, 2H, J=5.00 Hz), 3.51 (s, 2H), 3.65 (t, 2H, J=5.07 Hz), 6.93-6.98 (m, 1H, H-Aryl), 7.05-7.08 (m, 2H, H-Aryl), 7.25-7.30 (m, 1H, H-Aryl). MS (ESI+): m/z (%) 415 (100, [M+H]⁺), 437 (32, [M+Na]⁺), 851 (10, [2M+Na]⁺).

### Synthesis Example 25

### Preparation of Malonic Acid adamantan-1-yl ester 4-(2-methoxyphenyl)piperazine amide (31)

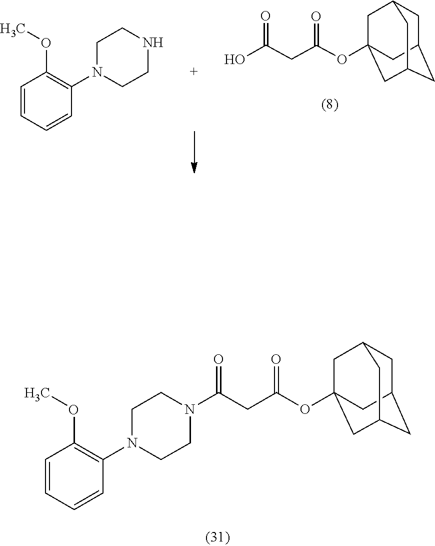 [SMILES: COc1ccccc1N1CCNCC1 ; O=C(O)CC(=O)OC12CC3CC(CC(C3)C1)C2 ; COc1ccccc1N1CCN(C(=O)CC(=O)OC23CC4CC(CC(C4)C2)C3)CC1]

The synthesis was carried out analogously to Synthesis Example 21 from malonic acid adamantan-1-ylester (8) (100 mg, 0.420 mmol), 1-(2-methoxyphenyl)piperazine (81 mg, 0.420 mmol) and triethylamine (TEA) (341 μl, 0.546 mmol) in DMF (4 ml) with 341 μl of a 1.6 M solution of T3P in ethyl acetate. Purification by column chromatography (dichloromethane/ethyl acetate 95:5→85:15) yielded the title compound (31) in the form of white crystals (130 mg).

¹H NMR (CDCl₃): δ=1.65 (m, 6H, adamantyl), 2.12-2.17 (m, 9H, adamantyl), 3.06 (dt, 4H, J=14.37, 10.21 Hz), 3.42 (s, 2H), 3.62 (t, 2H, J=5.04 Hz), 3.82 (t, 2H, J=5.10 Hz), 3.88 (s, 3H), 6.88-6.95 (m, 3H, H-Aryl), 7.02-7.06 (m, 1H, H-Aryl). MS (ESI+): m/z (%) 413 (100, [M+H]⁺), 435 (57, [M+Na]⁺), 847 (38, [2M+Na]⁺).

### Synthesis Example 26

### Preparation of Malonic Acid N-(3,5-dimethyladamantan-1-yl)amide (32)

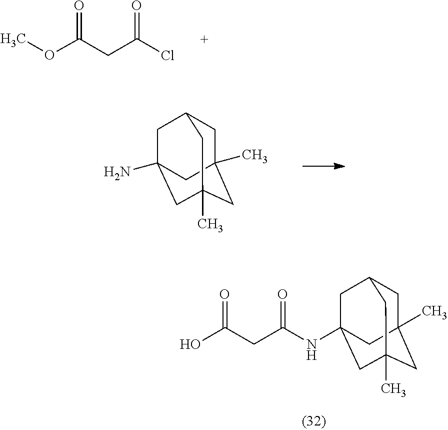

Malonic acid methyl ester chloride (173 mg, 1.27 mmol) was dissolved in 10 ml of dichloromethane under an inert gas atmosphere and cooled to −20° C., after which a solution of 3,5-dimethyl-1-adamantanamine (500 mg, 2.79 mmol) in 10 ml of dichloromethane was slowly added using a syringe. The reaction mixture was stirred at room temperature for 1.5 h, after which the solvent was removed and the residue was redissolved in ethyl acetate. The organic phase was washed with 1 N HCl and a saturated aqueous NaHCO₃ solution, dried over MgSO₄, filtered and evaporated to dryness. The residue, malonic acid methyl ester N-(3,5-dimethyladamantan-1-yl)amide, was dissolved in a mixture of THE and H₂O (2:1), to which 6 ml of 1 M aqueous NaOH were added and stirred at room temperature. After 1 h, the THF was removed on the rotary evaporator, and the remaining aqueous solution was acidified with 2 N HCl and extracted with ethyl acetate. The organic phase was dried over MgSO₄, filtered and evaporated to dryness, yielding the title compound (32) in the form of white crystals (260 mg).

¹H NMR (DMSO-d₆): δ=0.80 (s, 6H), 1.08 (s, 2H), 1.26 (qd, 4H, J=6.18, 2.86 Hz), 1.55 (s, 4H), 1.72 (d, 2H, J=2.97 Hz), 2.05 (quint, 1H, J=3.10 Hz), 3.04 (s, 2H), 7.56 (brs, 1H), 12.39 (brs, 1H). MS (ESI+): m/z (%) 266 (100, [M+H]⁺), 288 (9, [M+Na]⁺).

### Synthesis Example 27

### Preparation of N-(3,5-dimethyladamantan-1-yl)-N′-(3-aminopropyl)malonic Acid Diamide (33)

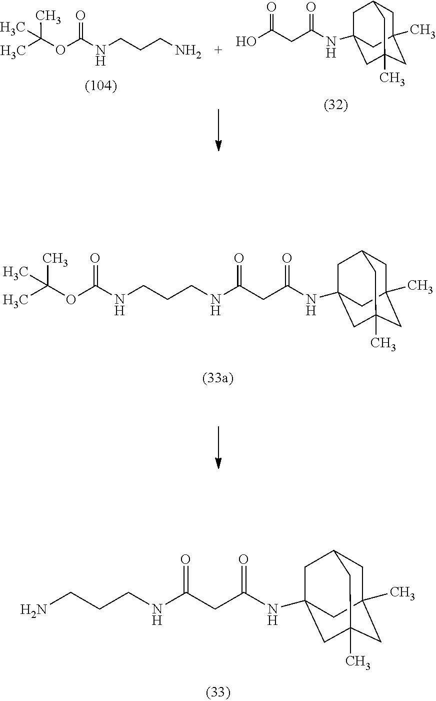 [SMILES: CC(C)(C)OC(=O)NCCCN ; CC12CC3CC(C)(C1)CC(NC(=O)CC(=O)O)(C3)C2 ; CC12CC3CC(C)(C1)CC(NC(=O)CC(=O)NCCCNC(=O)OC(C)(C)C)(C3)C2 ; CC12CC3CC(C)(C1)CC(NC(=O)CC(=O)NCCCN)(C3)C2]

The synthesis was carried out analogously to Synthesis Example 21 from malonic acid N-(3,5-dimethyladamantan-1-yl)amide (32) (100 mg, 0.377 mmol), 3-aminopropyl carbamic acid tert-butyl ester (104) (66 mg, 0.377 mmol) and triethylamine (TEA) (105 μl, 0.754 mmol) in DMF (4 ml) with 306 μl of a 1.6 M solution of T3P in ethyl acetate. Purification by column chromatography (dichloromethane/ethyl acetate 80:20→50:50) yielded the Boc-protected title compound (33a) in the form of a colorless oil (110 mg).

MS (ESI+): m/z (%) 422 (100, [M+H]⁺), 444 (100, [M+Na]⁺), 865 (90, [2M+Na]⁺).

The residue (110 mg, 0.261 mmol) thus obtained was dissolved in 30 ml of dichloromethane, after which 402 μl of trifluoroacetic acid (TFA) were added and the mixture was stirred for 4 h at room temperature. Subsequently, 60 ml of a saturated aqueous NaHCO₃ solution were added, and the mixture was extracted with dichloromethane. The organic phase was dried over MgSO₄, filtered and evaporated to dryness, yielding the title compound (33) in the form of a yellowish oil (76 mg).

¹H NMR (DMSO-d6): δ=0.79 (s, 6H), 1.08-1.10 (m, 3H), 1.22-1.31 (m, 4H), 1.54 (s, 4H), 1.65 (quint, 2H, J=6.86 Hz), 1.72 (d, 2H, J=2.48 Hz), 2.05 (m, 1H), 2.77 (t, 2H, J=7.30 Hz), 2.96 (s, 2H), 3.11 (q, 2H, J=6.36 Hz). MS (ESI+): m/z (%) 322 (100, [M+H]⁺), 344 (76, [M+Na]⁺), 643 (70, [2M+H]⁺).

### Synthesis Example 28

### Preparation of N-(3,5-dimethyladamantan-1-yl)-N′-(3-morpholinopropyl)malonic Acid Diamide (34)

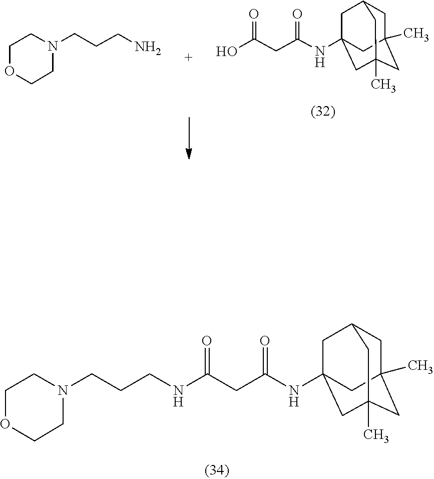 [SMILES: NCCCN1CCOCC1 ; CC12CC3CC(C)(C1)CC(NC(=O)CC(=O)O)(C3)C2 ; CC12CC3CC(C)(C1)CC(NC(=O)CC(=O)NCCCN1CCOCC1)(C3)C2]

The synthesis was carried out analogously to Synthesis Example 21 from malonic acid N-(3,5-dimethyl)adamantan-1-yl amide (32) (48 mg, 0.181 mmol), 3-morpholinopropylamine (26 mg, 0.181 mmol) and triethylamine (TEA) (51 μl, 0.362 mmol) in DMF (2 ml) with 147 μl of a 1.6 M solution of T3P in ethyl acetate. Purification by column chromatography (ethyl acetate→ethyl acetate/methanol 85:15) yielded the title compound (34) in the form of a colorless oil (50 mg).

¹H NMR (CDCl₃): δ=0.84 (s, 6H), 1.11-1.19 (m, 2H), 1.25-1.39 (m, 4H), 1.60-1.72 (m, 6H), 1.83 (d, 2H, J=2.95 Hz), 2.14 (quint, 1H, J=3.15 Hz), 2.40-2.44 (m, 6H), 3.02 (s, 2H), 3.34 (q, 2H, J=6.14 Hz), 3.71 (t, 4H, J=4.67 Hz), 6.64 (brs, 1H), 7.64 (brs, 1H). MS (ESI+): m/z (%) 392 (100, [M+H]⁺), 414 (24, [M+Na]⁺), 805 (22, [2M+Na]⁺).

### Synthesis Example 29

### Preparation of N-(3,5-dimethyladamantan-1-yl)-N′-piperazine Malonic Acid Diamide (35)

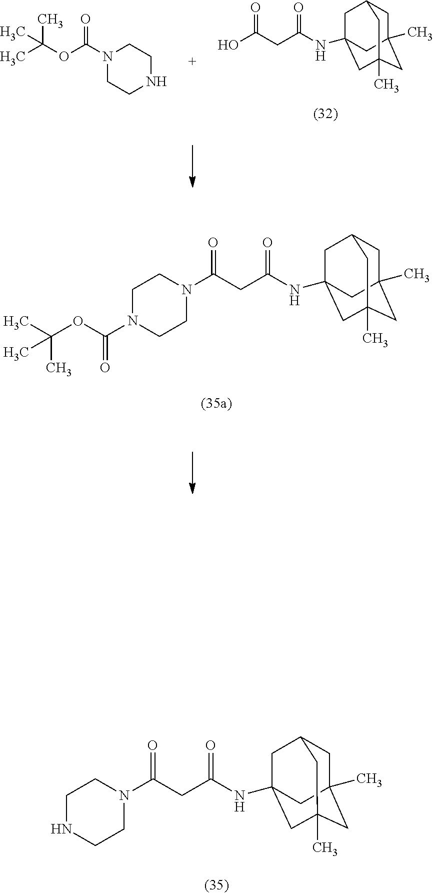 [SMILES: CC(C)(C)OC(=O)N1CCNCC1 ; CC12CC3CC(C)(C1)CC(NC(=O)CC(=O)O)(C3)C2 ; CC12CC3CC(C)(C1)CC(NC(=O)CC(=O)N1CCN(C(=O)OC(C)(C)C)CC1)(C3)C2 ; CC12CC3CC(C)(C1)CC(NC(=O)CC(=O)N1CCNCC1)(C3)C2]

The synthesis was carried out analogously to Synthesis Example 4 from malonic acid N-(3,5-dimethyl)adamantan-1-yl amide (32) (100 mg, 0.358 mmol), 1-piperazine carbamic acid tert-butyl ester (67 mg, 0.358 mmol) and triethylamine (100 μl, 0.716 mmol) in DMF (5 ml) with 291 μl of a 1.6 M solution of T3P in ethyl acetate. Purification of the residue obtained after extraction with ethyl acetate by column chromatography (dichloromethane/ethyl acetate 80:20→50:50) yielded the Boc-protected title compound (35a) in the form of a colorless oil (150 mg).

¹H NMR (CDCl₃): δ=0.83 (s, 6H), 1.10-1.19 (m, 2H), 1.25-1.30 (m, 2H), 1.35-1.39 (m, 2H), 1.46 (s, 9H, H−Boc), 1.59-1.68 (m, 5H), 1.83 (d, 2H, J=2.68 Hz), 2.13 (quint, 1H, J=3.15 Hz), 3.23 (s, 2H), 3.40-3.47 (m, 4H), 3.51-3.54 (m, 2H), 3.58-3.61 (m, 2H). MS (ESI+): m/z (%) 434 (100, [M+H]⁺), 456 (100, [M+Na]⁺), 867 (100, [2M+H]⁺), 889 (100, [2M+Na]⁺).

The product thus obtained (150 mg, 0.346 mmol) was deprotected analogously to Synthesis Example 4, yielding the hydrochloride of the title compound (35) in the form of a white solid (125 mg).

¹H NMR (DMSO-d₆): δ=0.79 (s, 6H), 1.08 (s, 2H), 1.21-1.31 (m, 4H), 1.54 (s, 4H), 1.72 (s, 2H, J=2.84 Hz), 2.05 (m, 1H), 2.99 (brs, 2H), 3.09 (brs, 2H), 3.63-3.67 (m, 4H), 7.66 (brs, 1H), 9.30 (brs, 1H). MS (ESI+): m/z (%) 334 (100, [M+H]⁺), 356 (31, [M+Na]⁺), 689 (32, [2M+Na]⁺).

### Synthesis Example 30

### Preparation of N-(3,5-dimethyladamantan-1-yl)-N′-4-(3-trifluoromethylbenzyl)piperazine Malonic Acid Diamide (36)

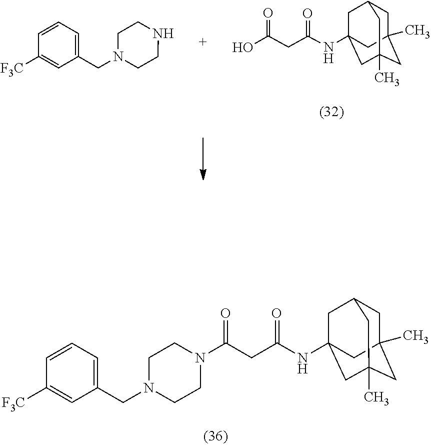 [SMILES: FC(F)(F)c1cccc(CN2CCNCC2)c1 ; CC12CC3CC(C)(C1)CC(NC(=O)CC(=O)O)(C3)C2 ; CC12CC3CC(C)(C1)CC(NC(=O)CC(=O)N1CCN(Cc4cccc(C(F)(F)F)c4)CC1)(C3)C2]

The synthesis was carried out analogously to Synthesis Example 21 from malonic acid N-(3,5-dimethyladamantan-1-yl)amide (32) (95 mg, 0.358 mmol), 1-(3-trifluoromethyl benzyl)piperazine (87 mg, 0.357 mmol) and triethylamine (TEA) (100 μl, 0.714 mmol) in DMF (2 ml) with 290 μl of a 1.6 M solution of T3P in ethyl acetate. Purification by column chromatography (dichloromethane/ethyl acetate 80:20→40:60) yielded the title compound (36) in the form of a colorless oil (110 mg).

¹H NMR (CDCl₃): δ=0.84 (s, 6H), 1.10-1.20 (m, 2H), 1.25-1.30 (m, 2H), 1.36-1.39 (m, 2H), 1.64 (q, 4H, J=11.77 Hz), 1.83 (brs, 2H), 2.13 (quint, 1H, J=3.13 Hz), 2.43 (brs, 4H), 3.21 (s, 2H), 3.56-3.65 (m, 6H), 7.42-7.59 (m, 4H, H-Aryl). MS (ESI+): m/z (%) 492 (100, [M+H]⁺), 514 (85, [M+Na]⁺), 983 (8, [2M+H]⁺).

### Synthesis Example 31

### Preparation of N-(3,5-dimethyladamantan-1-yl)-N′-4-(3-fluorobenzyl)piperazine Malonic Acid Diamide (37)

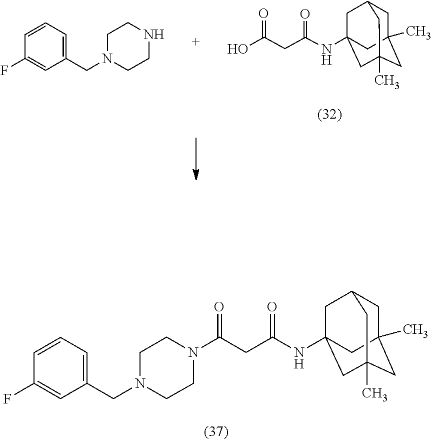 [SMILES: Fc1cccc(CN2CCNCC2)c1 ; CC12CC3CC(C)(C1)CC(NC(=O)CC(=O)O)(C3)C2 ; CC12CC3CC(C)(C1)CC(NC(=O)CC(=O)N1CCN(Cc4cccc(F)c4)CC1)(C3)C2]

The synthesis was carried out analogously to Synthesis Example 21 from malonic acid N-(3,5-dimethyladamantan-1-yl)amide (32) (95 mg, 0.358 mmol), 1-(3-fluorobenzyl)piperazine (70 mg, 0.358 mmol) and triethylamine (TEA) (100 μl, 0.716 mmol) in DMF (2 ml) with 290 μl of a 1.6 M solution of T3P in ethyl acetate. Purification by column chromatography (dichloromethane/ethyl acetate 50:50→ethyl acetate) yielded the title compound (37) in the form of a colorless oil (107 mg).

¹H NMR (CDCl₃): δ=0.84 (s, 6H), 1.10-1.20 (m, 2H), 1.25-1.30 (m, 2H), 1.36-1.40 (m, 2H), 1.64 (q, 4H, J=11.47 Hz), 1.83 (d, 2H, J=2.29 Hz), 2.13 (quint, 1H, J=3.15 Hz), 2.43 (brs, 4H), 3.21 (s, 2H), 3.51-3.63 (m, 6H), 6.94-7.30 (m, 4H, H-Aryl). MS (ESI+): m/z (%) 442 (100, [M+H]⁺), 464 (75, [M+Na]⁺), 905 (100, [2M+Na]⁺).

### Synthesis Example 32

### Preparation of N-(3,5-dimethyladamantan-1-yl)-N′-4-(2-methoxyphenyl)piperazine Malonic Acid Diamide (38)

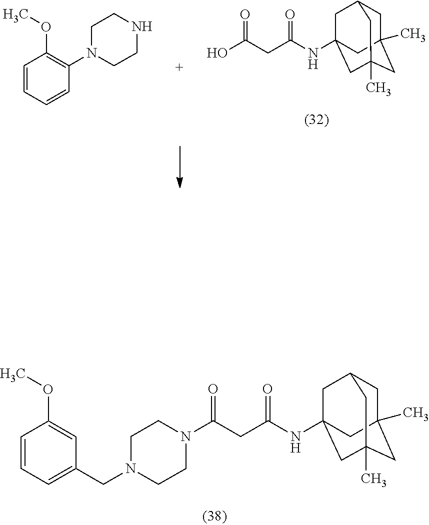 [SMILES: COc1ccccc1N1CCNCC1 ; CC12CC3CC(C)(C1)CC(NC(=O)CC(=O)O)(C3)C2 ; COc1cccc(CN2CCN(C(=O)CC(=O)NC34CC5CC(C)(CC(C)(C5)C3)C4)CC2)c1]

The synthesis was carried out analogously to Synthesis Example 21 from malonic acid N-(3,5-dimethyladamantan-1-yl)amide (32) (95 mg, 0.358 mmol), 1-(2-methoxyphenyl)piperazine (69 mg, 0.358 mmol) and triethylamine (TEA) (100 ml, 0.716 mmol) in DMF (2 ml) with 290 μl of a 1.6 M solution of T3P in ethyl acetate. Purification by column chromatography (dichloromethane/ethyl acetate 90:10→50:50) yielded the title compound (38) in the form of a white foam (120 mg).

¹H NMR (CDCl₃): δ=0.84 (s, 6H), 1.10-1.19 (m, 2H), 1.26-1.29 (m, 2H), 1.36-1.40 (m, 2H), 1.61-1.70 (m, 5H), 1.85 (d, 2H, J=2.76 Hz), 2.13 (quint, 1H, J=3.15 Hz), 3.05 (brs, 4H), 3.27 (s, 2H), 3.74 (brs, 2H), 3.82 (brs, 2H), 3.88 (s, 3H), 6.88-7.05 (m, 4H, H-Aryl). MS (ESI+): m/z (%) 440 (100, [M+H]⁺), 462 (77, [M+Na]⁺), 901 (84, [2M+Na]⁺).

### Synthesis Example 33

### Preparation of N-(3,5-dimethyladamantan-1-yl)-N′-4-(4-methoxyphenyl)piperazine Malonic Acid Diamide (39)

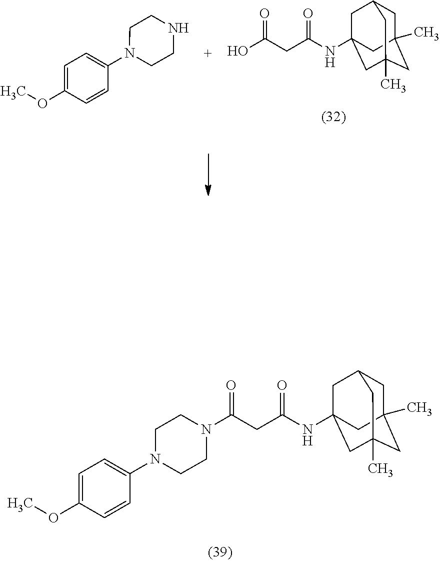

The synthesis was carried out analogously to Synthesis Example 21 from malonic acid N-(3,5-dimethyladamantan-1-yl)amide (32) (95 mg, 0.358 mmol), 1-(4-methoxyphenyl)piperazine (69 mg, 0.358 mmol) and triethylamine (TEA) (100 ml, 0.716 mmol) in DMF (2 ml) with 290 μl of a 1.6 M solution of T3P in ethyl acetate. Purification by column chromatography (dichloromethane/ethyl acetate 90:10→50:50) yielded the title compound (38) in the form of a white solid (113 mg).

¹H NMR (CDCl₃): δ=0.84 (s, 6H), 1.10-1.19 (m, 2H), 1.26-1.29 (m, 2H), 1.35-1.40 (m, 2H), 1.60-1.69 (m, 5H), 1.84 (d, 2H, J=2.75 Hz), 2.13 (quint, 1H, J=3.14 Hz), 3.05 (brs, 4H), 3.27 (s, 2H), 3.77 (s, 3H), 3.71-3.78 (m, 4H), 6.84-6.91 (m, 4H, H-Aryl). MS (ESI+): m/z (%) 440 (100, [M+H]⁺), 462 (100, [M+Na]⁺), 901 (100, [2M+Na]⁺).

### Synthesis Example 34

### Preparation of N-(3,5-dimethyladamantan-1-yl)-N′-4-(2,4-dimethoxyphenyl)piperazine Malonic Acid Diamide (40)

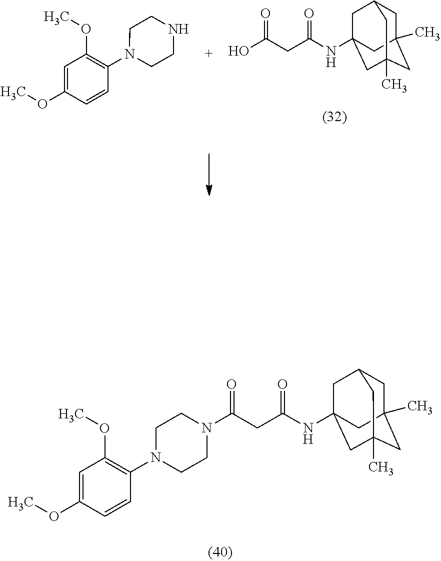

The synthesis was carried out analogously to Synthesis Example 21 from malonic acid N-(3,5-dimethyladamantan-1-yl)amide (32) (85 mg, 0.320 mmol), 1-(2,4-dimethoxyphenyl)piperazine (71 mg, 0.320 mmol) and triethylamine (TEA) (90 ml, 0.640 mmol) in DMF (2 ml) with 240 μl of a 1.6 M solution of T3P in ethyl acetate. Purification by column chromatography (dichloromethane/ethyl acetate 90:10→40:60) yielded the title compound (40) in the form of an off-white solid (120 mg).

¹H NMR (CDCl₃): δ=0.84 (s, 6H), 1.10-1.20 (m, 2H), 1.26-1.29 (m, 2H), 1.35-1.40 (m, 2H), 1.61-1.70 (m, 5H), 1.85 (d, 2H, J=2.71 Hz), 2.13 (quint, 1H, J=3.13 Hz), 2.96 (brs, 4H), 3.26 (s, 2H), 3.71 (brs, 2H), 3.78 (s, 3H), 3.80 (brs, 2H), 6.42 (dd, 2H, J=8.61 Hz, 2.55 Hz), 6.49 (d, 2H, J=2.65 Hz), 6.82 (d, 2H, J=8.43 Hz). MS (ESI+): m/z (%) 470 (100, [M+H]⁺, 492 (87, [M+Na]⁺), 961 (14, [2M+Na]⁺).

### Synthesis Example 35

### Preparation of N-(3,5-dimethyladamantan-1-yl)-N′-4-(2-morpholino-2-oxoethyl)piperazine Malonic Acid Diamide (41)

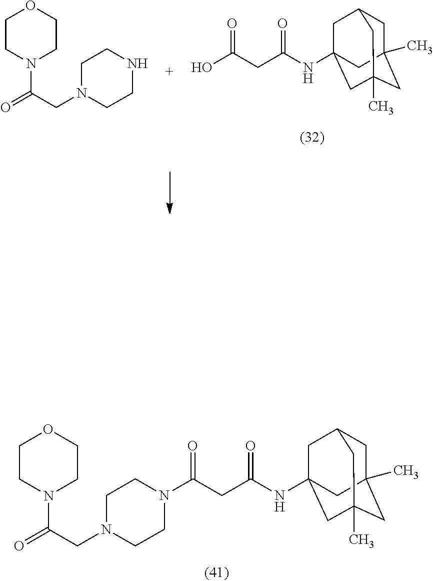 [SMILES: O=C(CN1CCNCC1)N1CCOCC1 ; CC12CC3CC(C)(C1)CC(NC(=O)CC(=O)O)(C3)C2 ; CC12CC3CC(C)(C1)CC(NC(=O)CC(=O)N1CCN(CC(=O)N4CCOCC4)CC1)(C3)C2]

The synthesis was carried out analogously to Synthesis Example 21 from malonic acid N-(3,5-dimethyladamantan-1-yl)amide (32) (68 mg, 0.256 mmol), 1-morpholino-2-(piperazine-1-yl)ethanon (55 mg, 0.256 mmol) and triethylamine (TEA) (72 ml, 0.512 mmol) in DMF (2 ml) with 208 μl of a 1.6 M solution of T3P in ethyl acetate. Purification by column chromatography (dichloromethane/ethyl acetate 90:10) yielded the title compound (41) in the form of a white foam (94 mg).

¹H NMR (CDCl₃): δ=0.84 (s, 6H), 1.10-1.20 (m, 2H), 1.25-1.29 (m, 2H), 1.35-1.39 (m, 2H), 1.64 (q, 4H, J=11.88 Hz), 1.83 (d, 2H, J=2.47 Hz), 2.12 (quint, 1H, J=3.13 Hz), 2.63 (brs, 4H), 3.22 (s, 2H), 3.29 (brs, 2H), 3.54-3.71 (m, 11H). MS (ESI+): m/z (%) 461 (100, [M+H]⁺), 483 (100, [M+Na]⁺), 921 (78, [2M+H]⁺), 943 (58, [2M+Na]⁺).

### Examples 1 to 41, Comparative Examples 1 to 8—Alzheimer Testing

The substances (1) to (41) and (101) to (108), prepared in the above synthesis examples or purchased, were tested for their potential suitability as an agent for treating Alzheimer's disease in a 96-well procedure in the initially mentioned screening assay using the nematode Caenorhabditis elegans (Pretsch et al., see above) in a standardized β-amyloid test system using the transgenic C. elegans strain CL2659. For this purpose, stage L3 worm larvae were used at a density of 10-20 worms per well. The expression of β-amyloid in the muscle cells was induced by increasing the temperature, and the associated paralysis was recorded at regular intervals until the end of the test after 48 hours. In this model, compared to a control substance, a significant delay in larval paralysis caused by neurotoxically active β-amyloid should be observed with active substances at concentrations that reduce β-amyloid expression.

For this purpose, the above substances were tested at concentrations of 1 mg/ml, 100 μg/ml, and 10 μg/ml in comparison with quercetin as a negative control. The test results are shown in Table 1 below, where the concentration range (in μg/ml) in which a delay in paralysis was observed (“Alzheimer's”) is indicated for the examples according to the invention, while no effect was observed for the comparative examples, even concentrations of 1 mg/ml.

### Examples 42 to 82, Comparative Examples 9 to 16—Parkinson Testing

The same substances as above were tested in a 96-well procedure in the initially mentioned screening assay using the nematode Caenorhabditis elegans (Pretsch et al., see above) in a standardized α-synuclein test system using the transgenic C. elegans strain NL5901 for their potential suitability as an agent for treating Parkinson's disease. This worm strain expresses human α-synuclein bound to green fluorescent protein (GFP) in all muscle cells. GFP labeling allows a direct detection of expressed protein levels using a multiplate reader for 24 to 48 hours. In this test model, compared to a control substance, a significant reduction in the expression of neurotoxic α-synuclein should be observed with active substances at concentrations at which there is a reduction in α-synuclein expression.

For this purpose, the substances were again tested at concentrations of 1 mg/ml, 100 μg/ml, and 10 μg/ml, but this time in comparison to the initially mentioned Parkinson therapeutic agent levodopa as a control. The test results are also shown in the table overleaf, where again the concentration range (in μg/ml) in which a reduction in α-synuclein expression was detectable (“Parkinson's”) is indicated for the examples according to the invention, while again no effect was observed for the comparative examples in this test, even at a concentration of 1 mg/ml.

| Compd. | | | | |
|---|---|---|---|---|
| No. | Chemical name | Alzheimer | Parkinson | Score |
| (1) | Malonic acid | >100 | >100 | + |
| (2) | Malonic acid diamide | >10 | >10 | ++ |
| (3) | Disodium malonate | >10 | >10 | ++ |
| (4) | Malonic acid dimethyl ester | >10 | >10 | ++ |
| (5) | Potassium methyl malonate | >10 | >10 | ++ |
| (6) | Malonic acid methyl ester amide | >100 | >100 | + |
| (7) | N-(3-Aminopropyl)malonic acid amide | >100 | >100 | + |
| (8) | Malonic acid adamantyl ester | >100 | >100 | + |
| (9) | Malonic acid adamantan-1-yl ethyl ester | >10 | >10 | ++ |
| (10) | Malonic acid adamantan-1-yl ester-N-(3-aminopropyl)amide | >10 | >10 | ++ |
| (11) | Malonic acid adamantan-1-yl ester-N-(3-aminopropyl)-N-methylamide | >10 | >10 | ++ |
| (12) | Malonic acid adamantan-1-yl ester piperazine amide | >10 | >10 | ++ |
| (13) | Malonic acid adamantan-1-yl ester-4-(4-aminobutyryl)piperazine amide | >10 | >10 | ++ |
| (14) | Malonic acid adamantan-1-yl ester-4-aminopiperidine amide | >10 | >10 | ++ |
| (15) | Malonic acid adamantan-1-yl ester-(4-(4-aminobutyryl)amino)piperidine | >10 | >10 | ++ |
| | amide | | | |
| (16) | Malonic acid methyl ester N-(adamantan-1-yl)amide | >10 | >10 | ++ |
| (17) | N-(Adamantan-1-yl)malonic acid amide | >10 | >10 | ++ |
| (18) | N-Adamantan-1-yl-N′-(3-aminopropyl)malonic acid diamide | >100 | >100 | + |
| (19) | N-Adamantan-1-yl-N′-(3-aminopropyl)-N′-methy Imalonic acid diamide | >10 | >10 | ++ |
| (20) | N-Adamantan-1-yl-N′-piperazine malonic acid diamide | >10 | >10 | ++ |
| (21) | N-Adamantan-1-yl-N′-(4-(4-aminobutyryl)piperazine)malonic acid | >10 | >10 | ++ |
| | diamide | | | |
| (22) | N-(3-Aminopropyl)-N′-morpholine malonic acid diamide | >10 | >10 | ++ |
| (23) | N-(3-Aminopropyl)-N′-piperidine malonic acid diamide | >10 | >10 | ++ |
| (24) | N-(3-Aminopropyl)-N′-decahydroquinoline malonic acid diamide | >10 | >10 | ++ |
| (25) | N-(3-Aminopropyl)-N′-decahydroisoquinoline malonic acid diamide | >10 | >10 | ++ |
| (26) | N-(3-Aminopropyl)-N′-(pinane-10-yl)malonic acid diamide | >10 | >10 | ++ |
| (27) | Malonic acid adamantan-1-yl ester N-(3-dimethylaminopropyl)amide | >10 | >10 | ++ |
| (28) | Malonic acid adamantan-1-yl ester N-(3-morpholinopropyl)amide | >10 | >10 | ++ |
| (29) | Malonic acid adamantan-1-yl ester 4-(3-trifluoromethylbenzyl)piperazine | >100 | >100 | ++ |
| | amide | | | |
| (30) | Malonic acid adamantan-1-yl ester 4-(3-fluorobenzyl)piperazine amide | >100 | >100 | ++ |
| (31) | Malonic acid adamantan-1-yl ester 4-(2-methoxyphenyl)piperazine | >100 | >100 | ++ |
| | amide | | | |
| (32) | Malonic acid N-(3,5-dimethyl)adamantan-1-yl amide | >10 | >10 | ++ |
| (33) | N-(3,5-Dimethyladamantan-1-yl)-N′-(3-aminopropyl)malonic acid | >10 | >10 | ++ |
| | diamide | | | |
| (34) | N-(3,5-Dimethyladamantan-1-yl)-N′-(3-morpholinopropyl)malonic acid | >10 | >10 | ++ |
| | diamide | | | |
| (35) | N-(3,5-Dimethyladamantan-1-yl)-N′-piperazine malonic acid diamide | >10 | >10 | ++ |
| (36) | N-(3,5-Dimethyladamantan-1-yl)-N′-4-(3- | >100 | >100 | ++ |
| | trifluoromethylbenzyl)piperazine malonic acid diamide | | | |
| (37) | N-(3,5-Dimethyladamantan-1-yl)-N′-4-(3-fluorobenzyl)piperazine | >100 | >100 | ++ |
| | malonic acid diamide | | | |
| (38) | N-(3,5-Dimethyladamantan-1-yl)-N′-4-(2-methoxyphenyl)piperazine | >100 | >100 | ++ |
| | malonic acid diamide | | | |
| (39) | N-(3,5-Dimethyladamantan-1-yl)-N′-4-(4-methoxyphenyl)piperazine | >100 | >100 | ++ |
| | malonic acid diamide | | | |
| (40) | N-(3,5-Dimethyladamantan-1-yl)-N′-4-(2,4-dimethoxyphenyl)piperazine | >100 | >100 | ++ |
| | malonic acid diamide | | | |
| (41) | N-(3,5-Dimethyladamantan-1-yl)-N′-4-(2-morpholino-2- | >10 | >10 | ++ |
| | oxoethyl)piperazine malonic acid diamide | | | |
| (101) | Dimethyl malonic acid | — | — | − |
| (102) | Methyl malonic acid dimethyl ester | — | — | − |
| (103) | 1-Adamantane carboxylic acid-N-(3-aminopropyl)amide | — | — | − |
| (104) | 3-Aminopropyl carbamic acid tert-butyl ester | — | — | − |
| (105) | 3-(Methylamino)propyl carbamic acid tert-butyl ester | — | — | − |
| (106) | 1-Adamantanol | — | — | − |
| (107) | 1-Adamantanamine | — | — | − |
| (108) | N-(3-Aminopropyl)-N′-tetrahydroquinoline malonic acid diamide | — | — | − |

The above test results clearly demonstrate that malonic acid and a plurality of derivatives thereof are promising potential agents against both Alzheimer's and Parkinson's disease, possibly also against Huntington's disease or prion diseases.

The results obtained for the various substitution patterns show that the active molecules can contain free carboxyl groups as well as salts, esters and substituted or unsubstituted amides without impairing their efficacy. The bulkiness of the substituents does not appear to play a significant role either, as both small (methyl, ethyl) and voluminous (e.g. dimethyl adamantyl or phenyl piperazinyl) groups showed comparable efficacy.

As is also shown by the comparative examples, this efficacy is apparently based in particular on the presence of an unsubstituted malonic acid moiety, as neither dimethyl malonic acid (101) nor dimethyl malonic acid dimethyl ester (102) proved to be effective. As the ineffectiveness of compounds (103) to (106) proves, the substituents contained in the derivatives of malonic acid effective according to the invention, such as the (dimethyl)adamantyl or the aminopropyl group, hardly influence the extent of the effectiveness. The ineffectiveness of the tetrahydroquinoline amide (108) cannot, of course, be attributed to the presence of the aromatic substituent, especially since although the non-aromatic decahydroquinoline amide (24) and its isomer, the decahydroisoquinoline amide (25), were already effective at a concentration >10 μg/ml, all phenylpiperazine amide derivatives also tested positive.

In addition, the dilution factor of 1:10 selected in the test series leaves a wide margin for the actually effective concentrations. This does not, for example, rule out the possibility that a compound that is already effective at a concentration >10 μg/ml and therefore rated “++” may only be effective at a concentration just below the limit value of 100 μg/ml, e.g., from 90 μg/ml up, while the efficacy limit of a compound that is only effective above 100 μg/ml and therefore rated “+” may be just above the limit value. Such compounds can actually differ in their efficacy by only a few percent.

It should also be borne in mind that the quantities of the respective compound actually available in the tests, i.e., the molar quantities, of course depend on the molecular weight, which varies within wide limits. For example, free malonic acid (1) (104.06 g/mol) and malonic acid diamide (2) (102.09 g/mol) have only a little more than a fifth of the molecular weight of N-(3,5-dimethyladamantan-1-yl)-N′-4-(3-trifluoromethylbenzyl)piperazine malonic acid diamide (36) (491.60 g/mol). This factor of approximately 5 already anticipates almost half of the dilution factor of 1:10—and yet malonic acid itself and three derivatives thereof with comparatively low molecular weights were only effective from concentrations >100 μg/ml, while compound (41), which has the second highest molecular weight after the aromatic compound (36), namely 460.62 g/mol, was already effective from >10 μg/ml.

Further investigations regarding the optimization of the efficacy by means of further variations of the substitution pattern and the dilution factors in the tests are therefore currently the subject of research by the inventors.

The present invention thus provides a group of compounds that, due to their efficacy in the C. elegans nematode assay, should be suitable for the treatment of neurodegenerative diseases such as Alzheimer's and Parkinson's disease, as they are able to induce the endogenous biosynthesis of metallothioneins—even at extremely low concentrations.

1. A compound comprising a malonic acid or derivative thereof having formula (I): 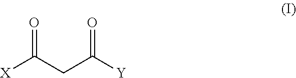
wherein X and Y are each independently selected from —O⁻M⁺, —OR₁, and —NR₂R₃, wherein M⁺ is selected from monovalent or polyvalent metal cations, and R₁, R₂, and R₃ are each independently selected from hydrogen and saturated or unsaturated, linear, branched, or cyclic hydrocarbon radicals having from 1 to 25 carbon atoms, wherein one or more carbon atoms are optionally substituted for O or N, wherein R₂ and R₃ are optionally connected to form a heterocyclic hydrocarbon radical together with the nitrogen atom, and
wherein the malonic acid or derivative thereof promotes metallothionein biosynthesis in a patient.

2. The compound according to claim 1, wherein both X and Y represent —O⁻M⁺, wherein each M⁺ is independently selected from mono- and polyvalent metal cations.

3. The compound according to claim 1, wherein R₁, R₂, and R₃ are each independently selected from hydrogen and saturated or unsaturated, linear, branched or cyclic carbon atoms having from 1 to 15 carbon atoms.

4. The compound according to claim 3, wherein R₁, R₂, and R₃ are each independently selected from hydrogen and saturated or unsaturated carbon atoms having from 1 to 10 carbon atoms.

5. The compound according to claim 1, wherein the compound is selected from the group consisting of malonic acid, malonic acid diamide, disodium malonate, malonic acid dimethyl ester, malonic acid methyl ester potassium salt, malonic acid methyl ester amide, N-(3-aminopropyl)malonic acid amide, malonic acid adamantan-1-yl ester, malonic acid adamantan-1-yl ethyl ester, malonic acid adamantan-1-yl ester N-(3-aminopropyl)amide, malonic acid adamantan-1-yl ester N-(3-aminopropyl)-N-methylamide, malonic acid adamantan-1-yl ester piperazine amide, malonic acid adamantan-1-yl ester 4-(4-aminobutyryl)piperazine amide, malonic acid adamantan-1-yl ester 4-aminopiperidine amide, malonic acid adamantan-1-yl ester (4-(4-aminobutyryl)amino)piperidine amide, malonic acid methyl ester N-(adamantan-1-yl)amide, N(adamantan-1-yl)malonic acid amide, N-adamantan-1-yl-N′-(3-aminopropyl)malonic acid diamide, N-adamantan-1-yl-N′-(3-aminopropyl)-N′-methyl malonic acid diamide, N-adamantan-1-yl-N′-piperazine malonic acid diamide, N-adamantan-1-yl-N′-(4-(4-aminobutyryl)piperazine)malonic acid diamide, N-(3-aminopropyl)-N′-morpholine malonic acid diamide, N-(3-aminopropyl)-N′-piperidine malonic acid diamide, N-(3-aminopropyl)-N′-decahydroquinoline malonic acid diamide, N-(3-aminopropyl)-N′-decahydroisoquinoline malonic acid diamide, N-(3-aminopropyl)-N′-(6,6-dimethylbicyclo[3.1.1]heptane-2-ylmethyl)malonic acid diamide, malonic acid adamantan-1-yl ester N-(3-dimethylaminopropyl)amide, malonic acid adamantan-1-yl ester N-(3-morpholinopropyl)amide, malonic acid adamantan-1-yl ester 4-(3-trifluoromethylbenzyl)piperazine amide, malonic acid adamantan-1-yl ester 4-(3-fluorobenzyl)piperazine amide, malonic acid adamantan-1-yl ester 4-(2-methoxyphenyl)piperazine amide, malonic acid N-(3,5-dimethyl)adamantan-1-yl amide, N-(3,5-dimethyladamantan-1-yl)-N′-(3-aminopropyl)malonic acid diamide, N-(3,5-dimethyladamantan-1-yl)-N′-(3-morpholinopropyl)malonic acid diamide, N-(3,5-dimethyladamantan-1-yl)-N′-piperazine malonic acid diamide, N-(3,5-dimethyladamantan-1-yl)-N′-4-(3-trifluoromethylbenzyl)piperazine malonic acid diamide, N-(3,5-dimethyladamantan-1-yl)-N′-4-(3-fluorobenzyl)piperazine malonic acid diamide, N-(3,5-dimethyladamantan-1-yl)-N′-4-(2-methoxyphenyl)piperazine malonic acid diamide, N-(3,5-dimethyladamantan-1-yl)-N′-4-(4-methoxyphenyl)piperazine malonic acid diamide, N-(3,5-dimethyladamantan-1-yl)-N′-4-(2,4-dimethoxyphenyl)piperazine malonic acid diamide, and N-(3,5-dimethyladamantan-1-yl)-N′-4-(2-morpholino-2-oxoethyl)piperazine malonic acid diamide.

6. The compound according to claim 1, wherein the promotion of metallothionein biosynthesis restores metal homeostasis and treats a neurodegenerative disease in the patient.

7. The compound according to claim 6, wherein the neurogenerative disease is selected from Alzheimer's disease, Parkinson's disease, and Huntington's disease.

8. The compound according to claim 7, wherein the neurogenerative disease is Alzheimer's or Parkinson's disease.

9. A pharmaceutical composition for therapeutically treating a human or animal body by promoting metallothionein biosynthesis in a patient, wherein the composition comprise the compound according to claim 1, at least one pharmaceutically acceptable excipient, and optionally one or more other pharmaceutically acceptable ingredients.

10. The pharmaceutical composition according to claim 9, wherein the compound treats a neurodegenerative disease.

11. The pharmaceutical composition according to claim 10, wherein the compound treats Alzheimer's or Parkinson's disease.

12. A method for promoting metallothionein biosynthesis in a patient, the method comprising administering to the patient the compound according to claim 1.

13. A method for treating a neurodegenerative disease in a patient, the method comprising administering to the patient the compound according to claim 1.

14. The method according to claim 13, wherein the neurodegenerative disease is selected from Alzheimer's disease, Parkinson's disease, and Huntington's disease.
